(19) United States
(12) Patent Application Publication
(10) Pub. No.: US 2015/0080235 A1
(43) Pub. Date: Mar. 19, 2015

(54) ASSAYS AND REAGENTS FOR DIAGNOSING TYPE 1 DIABETES

(71) Applicant: L2 Diagnostics, New Haven, CT (US)

(72) Inventor: Sahar Usmani-Brown, New Haven, CT (US)

(21) Appl. No.: 14/490,333

(22) Filed: Sep. 18, 2014

Related U.S. Application Data
(60) Provisional application No. 61/879,347, filed on Sep. 18, 2013.

(51) Int. Cl. C12Q 1/68

(52) U.S. Cl. CPC C12Q 1/6883; C12Q 2600/154; C12Q 2600/16; C12Q 2600/112
USPC 506/4; 506/16; 536/24.31

(57) ABSTRACT

Described herein are compositions and methods for diagnosing or monitoring type 1 diabetes.

### RELATED APPLICATIONS

This application claims the benefit of priority to U.S. Provisional Patent Application Ser. No. 61/879,347, filed Sep. 18, 2013; the contents of which are hereby incorporated by reference.

### BACKGROUND OF THE INVENTION

Type 1 diabetes is caused by the destruction of pancreatic β cells due to an autoimmune process, which occurs over years. By the time clinical symptoms appear, the mass of β cells is reduced by at least 70-80% (Cnop, M. et al., (2005) Diabetes December; 54 Suppl. 2:S97-107).

A nested PCR diagnostic for diabetes has been developed that detects the relative amount of circulating un-methylated β cell insulin DNA as a result of cell death (Akirav, E. M. et. al., (2011) Proc. Natl. Acad. Sci. 108: 19018-19023). However, nested PCR produces biases and artifacts.

Accordingly new methods for diagnosing and monitoring type 1 diabetes are needed.

### SUMMARY OF THE INVENTION

Featured herein are novel primers, probes and assays for diagnosing and monitoring type 1 diabetes.

The assays are highly sensitive and may be performed in a multiplexed fashion to diagnose diabetes before the onset of clinical symptoms and provide clinicians with a tool to decide for whom and when immune therapy might be useful.

### BRIEF DESCRIPTION OF THE FIGURE

FIG. 1 depicts the cloning of L2_UM DNA and L2_M DNA into the pUC57 plasmid. The underlined sequences highlight the regions of primers and probes.

### DETAILED DESCRIPTION OF THE INVENTION

Described herein are specific primers and probes, which can be used to measure the relative amounts of methylated and un-methylated insulin DNA in serum obtained from a human subject. The un-methylated form of the gene expresses functional insulin, while the methylated form does not express protein. Since β cells are the only significant source of insulin gene expression, the assay is able to measure epigenetically circulating un-methylated insulin DNA as a marker for β cell death. Methylated insulin DNA is used for normalizing the varying levels of DNA between individual specimens.

TABLE 1
List of primers and probes

| | |
|---|---|
| L2_KH1_FWD4 | GTGGTTTATATTTGGTGGA (SEQ ID NO. 3) |
| L2_KH1_REV4 | ATTAACTCACCCTACAAATC (SEQ ID NO. 4) |

The nucleotide sequence of the unmethylated insulin DNA detection probe is: ATTTAAGATTTGTTGGGAGGTAGAG (SEQ ID NO: 1) and the nucleotide sequence of the methylated insulin DNA detection probe is: ATTTAAGATTCGTCGGGAGGTAGAG (SEQ ID NO: 2). One of skill in the art could alter the probes by deleting, replacing of altering one or more nucleotides without substantially changing probe function.

The probes target a region of the human insulin gene with 2 CpG sites (i.e. regions of DNA where a cytosine nucleotide occurs next to a guanine nucleotide). When DNA is treated with bisulfite, the un-methylated CpG is converted to TG (thymine, guanine), while a methylated CpG site is protected from this change. The two probes described herein target either a methylated or un-methylated insulin gene.

Also described are double quencher probes specific
for un-methylated human insulin gene DNA detection
with fluorescein amidite (FAM):
L2_KH1_um
(SEQ ID NO: 3)
5′-/56-FAM/ATTTAAGAT/ZEN/TTGTTGGGAGGTAGAG/
3IABkFQ/-3′
and
for methylated human insulin gene detection
with hexachloro-fluoroscein (HEX):
L2_KH1_M
(SEQ ID NO: 4)
5′-/5HEX/ATTTAAGAT/ZEN/TCGTCGGGAGGTAGAG/
3IABkFQ/-3′

Use of these probes significantly reduces the background noise thereby increasing the noise to signal ratio.

The forward and reverse human insulin gene primers used in the examples are GTGGTTTATATTTGGTGGA (SEQ ID NO: 5) and ATTAACTCACCCTACAAATC (SEQ ID NO: 6). However, one of skill in the art may vary these sequences, e.g., by deleting, substituting or altering certain nucleotides without substantially impacting primer function.

The probes and primers described herein may be contacted with isolated and bisulfite-treated genomic DNA from an appropriate sample (e.g. serum, islet cells or peripheral blood mononuclear cells (PBMCs)). The reaction mixture may be loaded into a droplet generator. The droplets may be deposited on a plate and transferred into a polymerase chain reactor for amplification. The plate may then be transferred to a droplet reader for analysis of the data.

Use of the described probes and primers in a sensitive diagnostic assay, such as Droplet Digital Polymerase Chain Reaction (ddPCR), results in a highly specific and sensitive assay. In particular, as shown in the following examples, fewer than 13 copies were detected in a 25 ul PCR reaction with the target gene. In addition, in a recovery assay, where 5000 copies of L2_M plasmid representing bisulfite-treated methylated human insulin gene DNA were spiked with fewer than 10 copies of L2_UM plasmid representing bisulfite-treated un-methylated target DNA, L2_UM DNA was successfully detected.

As the results reported in the following example show, use of the described probes, primers and assays can be used to determine insulin DNA methylation status at low concentrations of DNA.

These primers and probes alone or in conjunction with instructions for use may be prepared as a kit for diagnosing or monitoring type 1 diabetes.

Table 1 lists additional probes and primers for amplification of methylation sensitive sites. Each assay has one set of probes, which can be used with 1 to 7 different sets of primers. Some probes will require modifications to the probes themselves to increase the annealing temperature and specificity such as—minor groove binder (MGB) modification, locked nucleic acid modification (LNA) and or Zen modification.

TABLE 1
List of Probes and Primers

| 5′-3′ | Type (5′-3′) | Sequence1 | Amplicon |
|---|---|---|---|
| | Probe- | AGAGTTTTGTTTTGTA (SEQ ID NO: 7) | |
| | Unmeth | | |
| | Probe_meth | AGAGTTTCGTTTTGTA (SEQ ID NO: 8) | |
| Primer Pair 1 | Forward | GGGATAGTAGTGTAA (SEQ ID NO: 9) | |
| | Primer | | |
| Primer Pair 1 | Reverse | CCTACTCACAACTAA (SEQ ID NO: 10) | 118 |
| | Primer | | |
| | Product | | |
| | Type | Sequence5 | Amplicon |
| | Probe- | TGGTTATTGGGTTTT (SEQ ID NO: 11) | |
| | Unmeth | | |
| | Probe_meth | TGGTTATCGGGTTTT (SEQ ID NO: 12) | |
| Primer Pair 1 | Forward | GGAAAGTGGTTTAGGTGAGGGTTT (SEQ | |
| | Primer | ID NO: 13) | |
| Primer Pair 1 | Reverse | CTCCTTAATCATCAACACCTCTTCCTC | 142 |
| | Primer | (SEQ ID NO: 14) | |
| | Product | | |
| Primer Pair 2 | Forward | TAGTTGTGAGTAGGGATAGGTT (SEQ ID | |
| | Primer | NO: 15) | |
| Primer Pair 2 | Reverse | AATCTCCTTAATCATCAACACCT (SEQ ID | 97 |
| | Primer | NO: 16) | |
| | Product | | |
| Primer Pair 3 | Forward | GGTTTAATGTGGAAAGTGGTTTAG (SEQ ID | |
| | Primer | NO: 17) | |
| Primer Pair 3 | Reverse | AACCATTTCCCTAATACTAAATCTATAA | 186 |
| | Primer | (SEQ ID NO: 18) | |
| | Product | | |
| Primer Pair 4 | Forward | TTTGGTTTAATGTGGAAAGT (SEQ ID NO: | |
| | Primer | 19) | |
| Primer Pair 4 | Reverse | CCAAACCATTTCCCTAATAC (SEQ ID NO: | 192 |
| | Primer | 20) | |
| | Product | | |
| Primer Pair 5 | Forward | GGGATAGTAGTGTAAAGAGTT (SEQ ID | |
| | Primer | NO: 21) | |
| Primer Pair 5 | Reverse | ACTACAATTTCCAAACCATTTC (SEQ ID | 245 |
| | Primer | NO: 22) | |
| | Product | | |
| | Type | Sequence6 | Amplicon |
| | Probe- | TTAATGATTTGTTGGTTTTGA (SEQ ID NO: | |
| | Unmeth | 23) | |
| | Probe_meth | TTAATGATTCGTTGGTTTTGA (SEQ ID NO: | |
| | | 24) | |
| Primer Pair 1 | Forward | GGAAAGTGGTTTAGGTGAGGGTTT (SEQ | |
| | Primer | ID NO: 25) | |
| Primer Pair 1 | Reverse | ATCTCCTTAATCATCAACACCTCTTCC | 144 |
| | Primer | (SEQ ID NO: 26) | |
| | Product | | |
| Primer Pair 2 | Forward | GTGAGTAGGGATAGGTTTGGTTAT (SEQ | |
| | Primer | ID NO: 27) | |
| Primer Pair 2 | Reverse | CCCTACCACCTAACCCATTAAA (SEQ ID | 197 |
| | Primer | NO: 28) | |
| | Product | | |
| Primer Pair 3 | Forward | GGATAGGTTTGGTTATTGGGTTT (SEQ ID | |
| | Primer | NO: 29) | |
| Primer Pair 3 | Reverse | TCCAAACCATTTCCCTAATACTAAAT (SEQ | 119 |
| | Primer | ID NO: 30) | |
| | Product | | |
| Primer Pair 4 | Forward | TAGTTGTGAGTAGGGATAGGT (SEQ ID | |
| | Primer | NO: 31) | |
| Primer Pair 4 | Reverse | AAACTACAATTTCCAAACCATTTC (SEQ ID | 143 |
| | Primer | NO: 32) | |
| | Product | | |
| Primer Pair 5 | Forward | TTTAATGTGGAAAGTGGTTTAG (SEQ ID | |
| | Primer | NO: 33) | |
| Primer Pair 5 | Reverse | AAATCTCCTTAATCATCAACAC (SEQ ID | 154 |
| | Primer | NO: 34) | |
| | Product | | |
| | Type | Sequence7 | Amplicon |
| | Probe- | AAGAGGTGTTGATGATTAAGGAGAT (SEQ ID | |
| | Unmeth | NO: 35) | |
| | Probe_meth | AAGAGGTGTTGACGATTAAGGAGAT (SEQ ID | |
| | | NO: 36) | |
| Primer Pair 1 | Forward | GTGAGTAGGGATAGGTTTGGTTAT (SEQ | |
| | Primer | ID NO: 37) | |
| Primer Pair 1 | Reverse | CCCTACCACCTAACCCATTAAA (SEQ ID | 197 |
| | Primer | NO: 38) | |
| | Product | | |
| Primer Pair 2 | Forward | GAAAGTGGTTTAGGTGAGGGTT (SEQ ID | |
| | Primer | NO: 39) | |
| Primer Pair 2 | Reverse | TCCAAACCATTTCCCTAATACTAAATCT | 179 |
| | Primer | (SEQ ID NO: 40) | |
| | Product | | |
| Primer Pair 3 | Forward | TAGTTGTGAGTAGGGATAGGT (SEQ ID | |
| | Primer | NO: 41) | |
| Primer Pair 3 | Reverse | AAACTACAATTTCCAAACCATTTC (SEQ ID | 143 |
| | Primer | NO: 42) | |
| | Product | | |
| Primer Pair 4 | Forward | GATAGGTTTGGTTATTGGGTTT (SEQ ID | |
| | Primer | NO: 43) | |
| Primer Pair 4 | Reverse | ATTTCCAAACCATTTCCCTAATA (SEQ ID | 121 |
| | Primer | NO: 44) | |
| | Product | | |
| Primer Pair 5 | Forward | TTTAATGATTTGTTGGTTT (SEQ ID NO: 45) | |
| | Primer | | |
| Primer Pair 5 | Reverse | CCCACTAACTTTATAATCTC (SEQ ID NO: | 201 |
| | Primer | 46) | |
| | Product | | |
| | Type | Sequence8 | Amplicon |
| | Probe- | AAATGGTTTGGAAATTGTA (SEQ ID NO: 47) | |
| | Unmeth | | |
| | Probe_meth | AAATGGTTCGGAAATTGTA (SEQ ID NO: 48) | |
| Primer Pair 1 | Forward | TGAGGAAGAGGTGTTGATGATT (SEQ ID | |
| | Primer | NO: 49) | |
| Primer Pair 1 | Reverse | CCCTACCACCTAACCCATTAAA (SEQ ID | 136 |
| | Primer | NO: 50) | |
| | Product | | |
| Primer Pair 2 | Forward | AGAGGTGTTGATGATTAAGGAGATT (SEQ | |
| | Primer | ID NO: 51) | |
| Primer Pair 2 | Reverse | ACCTCTTCTAATACAACCTATCCTAAA | 223 |
| | Primer | (SEQ ID NO: 52) | |
| | Product | | |
| Primer Pair 3 | Forward | AGTTGTGAGTAGGGATAGG (SEQ ID NO: | |
| | Primer | 53) | |
| Primer Pair 3 | Reverse | CCCACTAACTTTATAATCTCAAA (SEQ ID | 247 |
| | Primer | NO: 54) | |
| | Product | | |
| Primer Pair 4 | Forward | TTTGAGGAAGAGGTGTT (SEQ ID NO: 55) | |
| | Primer | | |
| Primer Pair 4 | Reverse | ACCTCTTCTAATACAACCTA (SEQ ID NO: | 231 |
| | Primer | 56) | |
| | Product | | |
| Primer Pair 5 | Forward | TTTATAGATTTAGTATTAGGG (SEQ ID NO: | |
| | Primer | 57) | |
| Primer Pair 5 | Reverse | CTACTTAATAACCTCTTCT (SEQ ID NO: 58) | 206 |
| | Primer | | |
| | Product | | |
| | Type | Sequence9 | Amplicon |
| | Probe- | TTAGGTGGTAGGG (SEQ ID NO: 59) | |
| | Unmeth | | |
| | Probe_meth | TTAGGTGGTAGGG (SEQ ID NO: 60) | |
| Primer Pair 1 | Reverse | AATAACCTCTTCTAATACAACCTATCCT | 183 |
| Primer Pair 1 | Primer | (SEQ ID NO: 61) | |
| | Product | | |
| Primer Pair 2 | Forward | AGTATTAGGGAAATGGTTTGGA (SEQ ID | |
| | Primer | NO: 62) | |
| Primer Pair 2 | Reverse | CAAACCTACTTAATAACCTCTTCTAAT | 200 |
| | Primer | (SEQ ID NO: 63) | |
| | Product | | |
| Primer Pair 3 | Forward | GAGGAAGAGGTGTTGATG (SEQ ID NO: 64) | |
| | Primer | | |
| Primer Pair 3 | Reverse | CCCACTAACTTTATAATCTCAAA (SEQ ID | 181 |
| | Primer | NO: 65) | |
| | Product | | |
| Primer Pair 4 | Forward | TTTAGTATTAGGGAAATG (SEQ ID NO: 66) | |
| | Primer | | |
| Primer Pair 4 | Reverse | ACCTACTTAATAACCTC (SEQ ID NO: 67) | 200 |
| | Primer | | |
| | Product | | |
| | Type | Sequence10 | Amplicon |
| | Probe- | AAGTTAGTGGGGGTTT (SEQ ID NO: 68) | |
| | Unmeth | | |
| | Probe_meth | AAGTTAGCGGGGGTTT (SEQ ID NO: 69) | |
| Primer Pair 1 | Forward | TTTAATGGGTTAGGTGGTAGGG (SEQ ID | |
| | Primer | NO: 70) | |
| Primer Pair 1 | Reverse | ACCTCTTCTAATACAACCTATCCTAAA | 115 |
| | Primer | (SEQ ID NO: 71) | |
| | Product | | |
| Primer Pair 2 | Forward | GGGAAATGGTTTGGAAATTGTAGTT (SEQ | |
| | Primer | ID NO: 72) | |
| Primer Pair 2 | Reverse | ACCTACTTAATAACCTCTTCTAATACAACC | 190 |
| | Primer | (SEQ ID NO: 73) | |
| | Product | | |
| Primer Pair 3 | Forward | GTATTAGGGAAATGGTTTGGAAAT (SEQ ID | |
| | Primer | NO: 74) | |
| Primer Pair 3 | Reverse | ACAAACCTACTTAATAACCTCTTCTA (SEQ | 200 |
| | Primer | ID NO: 75) | |
| | Product | | |
| Primer Pair 4 | Forward | AGTATTAGGGAAATGGT (SEQ ID NO: 76) | |
| | Primer | | |
| Primer Pair 4 | Reverse | CAAACCTACTTAATAACC (SEQ ID NO: 77) | 200 |
| | Primer | | |
| | Product | | |
| | Type | Sequence12 | Amplicon |
| | Probe- | TTTGTGTTAGGT (SEQ ID NO: 78) | |
| | Unmeth | | |
| | Probe_meth | TTTGCGTTAGGT (SEQ ID NO: 79) | |
| Primer Pair 1 | Forward | GGTTGTATTAGAAGAGGTTATTAAGTAGGT | |
| | Primer | (SEQ ID NO: 80) | |
| Primer Pair 1 | Reverse | CTTCACAAACCCAACCACATCC (SEQ ID | 131 |
| | Primer | NO: 81) | |
| | Product | | |
| Primer Pair 2 | Forward | TTAGGATAGGTTGTATTAGAAGAGGTTATT | |
| | Primer | (SEQ ID NO: 82) | |
| Primer Pair 2 | Reverse | CCAACCACATCCTCCCTACT (SEQ ID NO: | 129 |
| | Primer | 83) | |
| | Product | | |
| Primer Pair 3 | Forward | TAATGGGTTAGGTGGTAGGG (SEQ ID NO: | |
| | Primer | 84) | |
| Primer Pair 3 | Reverse | CCCACATACTTCACAAACC (SEQ ID NO: | 234 |
| | Primer | 85) | |
| | Product | | |
| Primer Pair 4 | Forward | AGGATAGGTTGTATTAGAAGA (SEQ ID NO: | |
| | Primer | 86) | |
| Primer Pair 4 | Reverse | AAATCCAACCACCCTAAA (SEQ ID NO: 87) | 91 |
| | Primer | | |
| | Product | | |
| Primer Pair 5 | Forward | TTGAGATTATAAAGTTAGTGG (SEQ ID NO: | |
| | Primer | 88) | |
| Primer Pair 5 | Reverse | AAACCAAATACCCTACC (SEQ ID NO: 89) | 227 |
| | Primer | | |
| | Product | | |
| | Type | Sequence13 | Amplicon |
| | Probe- | TAGGGAGGATGTGGTTGGGT (SEQ ID NO: | |
| | Unmeth | 90) | |
| | Probe_meth | TAGGGAGGACGTGGTTGGGT (SEQ ID NO: | |
| | | 91) | |
| Primer Pair 1 | Forward | GTGTTAGGTGGGTTTAGGA (SEQ ID NO: | |
| | Primer | 92) | |
| Primer Pair 1 | Reverse | AAACCAACAACACCAACAA (SEQ ID NO: | 223 |
| | Primer | 93) | |
| | Product | | |
| Primer Pair 2 | Forward | AGGATAGGTTGTATTAGAAGAGG (SEQ ID | |
| | Primer | NO: 94) | |
| Primer Pair 2 | Reverse | AAACCAAATACCCTACCTTAAA (SEQ ID | 182 |
| | Primer | NO: 95) | |
| | Product | | |
| Primer Pair 3 | Forward | TTAGGGTGGTTGGATTT (SEQ ID NO: 96) | |
| | Primer | | |
| Primer Pair 3 | Reverse | CACACAAATATTAATTCACAAA (SEQ ID | 250 |
| | Primer | NO: 97) | |
| | Product | | |
| | Type | Sequence14 | Amplicon |
| | Probe- | TTGTTGGTGTTGTTGG (SEQ ID NO: 98) | |
| | Unmeth | | |
| | Probe_meth | TTGTTGGCGTTGTTGG (SEQ ID NO: 99) | |
| Primer Pair 1 | Forward | GAGGATGTGGTTGGGTTTGTGA (SEQ ID | |
| | Primer | NO: 100) | |
| Primer Pair 1 | Reverse | AACTTCCACCAAATATAAACCACACAAATA | 231 |
| | Primer | (SEQ ID NO: 101) | |
| | Product | | |
| Primer Pair 2 | Forward | GGTTGGGTTTGTGAAGTATGTG (SEQ ID | |
| | Primer | NO: 102) | |
| Primer Pair 2 | Reverse | ACCAAATATAAACCACACAAATATTAATTC | 216 |
| | Primer | (SEQ ID NO: 103) | |
| | Product | | |
| Primer Pair 3 | Forward | TTGTAGTAGGGAGGATGTGG (SEQ ID NO: | |
| | Primer | 104) | |
| Primer Pair 3 | Reverse | AAACTTCCACCAAATATAAACCA (SEQ ID | 242 |
| | Primer | NO: 105) | |
| | Product | | |
| Primer Pair 4 | Forward | TTTAAGGTAGGGTATTTGGTTT (SEQ ID | |
| | Primer | NO: 106) | |
| Primer Pair 4 | Reverse | CCTCTACCTCCCAACAAATC (SEQ ID NO: | 248 |
| | Primer | 107) | |
| | Product | | |
| Primer Pair 5 | Forward | GTGTTAGGTGGGTTTAGGA (SEQ ID NO: | |
| | Primer | 108) | |
| Primer Pair 5 | Reverse | AAACTACAACTAAATCAAATCCC (SEQ ID | 250 |
| | Primer | NO: 109) | |
| | Product | | |
| Primer Pair 6 | Forward | TTTAAGGTAGGGTATTTGGTTT (SEQ ID | |
| | Primer | NO: 110) | |
| Primer Pair 6 | Reverse | ACCACACAAATATTAATTCACAAA (SEQ ID | 166 |
| | Primer | NO: 111) | |
| | Product | | |
| Primer Pair 7 | Forward | TTGTAGTAGGGAGGATGTGG (SEQ ID NO: | |
| | Primer | 112) | |
| Primer Pair 7 | Reverse | AAACTACAACTAAATCAAATCCC (SEQ ID | 200 |
| | Primer | NO: 113) | |
| | Product | | |
| | | Sequence15 | |
| | Probe- | TTAGTTGTAGTT (SEQ ID NO: 114) | |
| | Unmeth | | |
| | Probe_meth | TTAGTCGTAGTT (SEQ ID NO: 115) | |
| Primer Pair 1 | Forward | GGTTGGGTTTGTGAAGTATGTG (SEQ ID | |
| | Primer | NO: 116) | |
| Primer Pair 1 | Reverse | ACCAAATATAAACCACACAAATATTAATTC | 216 |
| | Primer | (SEQ ID NO: 117) | |
| | Product | | |
| Primer Pair 2 | Forward | TTGTAGTAGGGAGGATGTGG (SEQ ID NO: | |
| | Primer | 118) | |
| Primer Pair 2 | Reverse | AAACTTCCACCAAATATAAACCA (SEQ ID | 242 |
| | Primer | NO: 119) | |
| | Product | | |
| Primer Pair 3 | Forward | TTTGTTGGTGTTGTTGGTTT (SEQ ID NO: | |
| | Primer | 120) | |
| Primer Pair 3 | Reverse | CCTCTACCTCCCAACAAATC (SEQ ID NO: | 152 |
| | Primer | 121) | |
| | Product | | |
| Primer Pair 4 | Forward | TTTAAGGTAGGGTATTTGGTTT (SEQ ID | |
| | Primer | NO: 122) | |
| Primer Pair 4 | Reverse | ACCACACAAATATTAATTCACAAA (SEQ ID | 166 |
| | Primer | NO: 123) | |
| | Product | | |
| | Type | Sequence16 | Amplicon |
| | Probe- | TTGTGTGGTTTA (SEQ ID NO: 124) | |
| | Unmeth | | |
| | Probe_meth | TTGTGCGGTTTA (SEQ ID NO: 125) | |
| Primer Pair 1 | Forward | TTTGTTGGTGTTGTTGGTTT (SEQ ID NO: | |
| | Primer | 126) | |
| Primer Pair 1 | Reverse | CCTCTACCTCCCAACAAATC (SEQ ID NO: | 152 |
| | Primer | 127) | |
| | Product | | |
| Primer Pair 2 | Forward | GGGATTTGATTTAGTTGTAGTTT (SEQ ID | |
| | Primer | NO: 128) | |
| Primer Pair 2 | Reverse | CTCACCCTACAAATCCTCTAC (SEQ ID NO: | 142 |
| | Primer | 129) | |
| | Product | | |
| Primer Pair 3 | Forward | TTTAAGGTAGGGTATTTGGTT (SEQ ID NO: | |
| | Primer | 130) | |
| Primer Pair 3 | Reverse | ACCTCCCAACAAATCTTAAATA (SEQ ID | 243 |
| | Primer | NO: 131) | |
| | Product | | |
| Primer Pair 4 | Forward | GTTGGGTTTGTGAAGTA (SEQ ID NO: 132) | |
| | Primer | | |
| Primer Pair 4 | Reverse | CCCACACACTAAATAAA (SEQ ID NO: 133) | 240 |
| | Primer | | |
| | Product | | |
| | Type | Sequence17 | Amplicon |
| | Probe- | AGTGTGTGGGGAA (SEQ ID NO: 134) | |
| | Unmeth | | |
| | Probe_meth | AGTGTGCGGGGAA (SEQ ID NO: 135) | |
| Primer Pair 1 | Forward | ATTTGTGTGGTTTATATTTGGTGGAAGT | |
| | Primer | (SEQ ID NO: 136) | |
| Primer Pair 1 | Reverse | ACTCACCCTACAAATCCTCTACCT (SEQ ID | 108 |
| | Primer | NO: 137) | |
| | Product | | |
| Primer Pair 2 | Forward | TTGTGAATTAATATTTGTGTGGTTTATATT | |
| | Primer | (SEQ ID NO: 138) | |
| Primer Pair 2 | Reverse | TCCTCTACCTCCCAACAAATC (SEQ ID NO: | 106 |
| | Primer | 139) | |
| | Product | | |
| Primer Pair 3 | Forward | TTTGTTGGTGTTGTTGGTTT (SEQ ID NO: | |
| | Primer | 140) | |
| Primer Pair 3 | Reverse | TCTACCTCCCAACAAATCTTAAATA (SEQ | 150 |
| | Primer | ID NO: 141) | |
| | Product | | |
| Primer Pair 4 | Forward | TTTGTGAATTAATATTTGTGTGGT (SEQ ID | |
| | Primer | NO: 142) | |
| Primer Pair 4 | Reverse | CAATAAACAATTAACTCACCCTAC (SEQ ID | 134 |
| | Primer | NO: 143) | |
| | Product | | |
| Primer Pair 5 | Forward | GGGATTTGATTTAGTTGTAGTTT (SEQ ID | |
| | Primer | NO: 144) | |
| Primer Pair 5 | Reverse | CTAAATAACAACCTCCTACCC (SEQ ID NO: | 240 |
| | Primer | 145) | |
| | Product | | |
| | Type | Sequence18 | Amplicon |
| | Probe- | AGATTTGTTGGGAG (SEQ ID NO: 146) | |
| | Unmeth | | |
| | Probe_meth | AGATTCGTCGGGAG (SEQ ID NO: 147) | |
| Primer Pair 1 | Forward | ATTTGTGTGGTTTATATTTGGTGGAAGT | |
| | Primer | (SEQ ID NO: 148) | |
| Primer Pair 1 | Reverse | ACAATTAACTCACCCTACAAATCCTCTAC | 115 |
| | Primer | (SEQ ID NO: 149) | |
| | Product | | |
| Primer Pair 2 | Forward | TGAATTAATATTTGTGTGGTTTATATTTGG | |
| | Primer | (SEQ ID NO: 150) | |
| Primer Pair 2 | Reverse | ACAATAAACAATTAACTCACCCTACA (SEQ | 131 |
| | Primer | ID NO: 151) | |
| | Product | | |
| Primer Pair 3 | Forward | TTTGTGAATTAATATTTGTGTGGTTTA (SEQ | |
| | Primer | ID NO: 152) | |
| Primer Pair 3 | Reverse | CCTACTAAATAACAACCTCCTACC (SEQ ID | 222 |
| | Primer | NO: 153) | |
| | Product | | |
| Primer Pair 4 | Forward | TGTTGGTGTTGTTGGTTT (SEQ ID NO: 154) | |
| | Primer | | |
| Primer Pair 4 | Reverse | CCCTTCTACCCATACTAAATAAA (SEQ ID | 242 |
| | Primer | NO: 155) | |
| | Product | | |
| Primer Pair 5 | Forward | TTTGTGAATTAATATTTGTG (SEQ ID NO: | |
| | Primer | 156) | |
| Primer Pair 5 | Reverse | CCCTACTAAATAACAACC (SEQ ID NO: | 223 |
| | Primer | 157) | |
| | Product | | |
| | Type | Sequence20 | Amplicon |
| | Probe- | TTGGTTGTTTTT (SEQ ID NO: 158) | |
| | Unmeth | | |
| | Probe_meth | TTGGTCGTTTTT (SEQ ID NO: 159) | |
| Primer Pair 1 | Forward | GGTAGAGGATTTGTAGGGTGAGTTAAT | |
| | Primer | (SEQ ID NO: 160) | |
| Primer Pair 1 | Reverse | CCCTACTAAATAACAACCTCCTACCC (SEQ | 125 |
| | Primer | ID NO: 161) | |
| | Product | | |
| Primer Pair 2 | Forward | TTGTTGGGAGGTAGAGGATTTGTA (SEQ | |
| | Primer | ID NO: 162) | |
| Primer Pair 2 | Reverse | AAACCAACACCATCCTCAAACTAAA (SEQ | 238 |
| | Primer | ID NO: 163) | |
| | Product | | |
| Primer Pair 3 | Forward | AGGGTGAGTTAATTGTTTATTGTTGTTT | |
| | Primer | (SEQ ID NO: 164) | |
| Primer Pair 3 | Reverse | ACATACCCACCCTCTAATATATCTCAA | 250 |
| | Primer | (SEQ ID NO: 165) | |
| | Product | | |
| Primer Pair 4 | Forward | TAAGATTTGTTGGGAGGTAGAG (SEQ ID | |
| | Primer | NO: 166) | |
| Primer Pair 4 | Reverse | CCCTTCTACCCATACTAAATAAA (SEQ ID | 115 |
| | Primer | NO: 167) | |
| | Product | | |
| Primer Pair 5 | Forward | GTAGGGTGAGTTAATTGTTTATT (SEQ ID | |
| | Primer | NO: 168) | |
| Primer Pair 5 | Reverse | AACCAACACCATCCTCA (SEQ ID NO: 169) | 216 |
| | Primer | | |
| | Product | | |
| | Type | Sequence21 | Amplicon |
| | Probe- | TTTGGTGTTTTT (SEQ ID NO: 170) | |
| | Unmeth | | |
| | Probe_meth | TTTGGCGTTTTT (SEQ ID NO: 171) | |
| Primer Pair 1 | Forward | GGTAGAGGATTTGTAGGGTGAGTTAAT | |
| | Primer | (SEQ ID NO: 172) | |
| Primer Pair 1 | Reverse | CCCTACTAAATAACAACCTCCTACCC (SEQ | 125 |
| | Primer | ID NO: 173) | |
| | Product | | |
| Primer Pair 2 | Forward | TTGTTGGGAGGTAGAGGATTTGTA (SEQ | |
| | Primer | ID NO: 174) | |
| Primer Pair 2 | Reverse | AAACCAACACCATCCTCAAACTAAA (SEQ | 238 |
| | Primer | ID NO: 175) | |
| | Product | | |
| Primer Pair 3 | Forward | AGGGTGAGTTAATTGTTTATTGTTGTTT | |
| | Primer | (SEQ ID NO: 176) | |
| Primer Pair 3 | Reverse | ACATACCCACCCTCTAATATATCTCAA | 250 |
| | Primer | (SEQ ID NO: 177) | |
| | Product | | |
| Primer Pair 4 | Forward | AGATTTGTTGGGAGGTAGAG (SEQ ID NO: | |
| | Primer | 180) | |
| Primer Pair 4 | Reverse | AAACCAACACCATCCTCA (SEQ ID NO: | 242 |
| | Primer | 181) | |
| | Product | | |
| Primer Pair 5 | Forward | GTAGGGTGAGTTAATTGTTTAT (SEQ ID | |
| | Primer | NO: 182) | |
| Primer Pair 5 | Reverse | CCCTTCTACCCATACTAAAT (SEQ ID NO: | 88 |
| | Primer | 183) | |
| | Product | | |
| | Type | Sequence22 | Amplicon |
| | Probe- | TGGTTATGTTTTAA (SEQ ID NO: 184) | |
| | Unmeth | | |
| | Probe_meth | TGGTTACGTTTTAA (SEQ ID NO: 185) | |
| Primer Pair 1 | Forward | GGGTAGGAGGTTGTTATTTAGTAGGG | |
| | Primer | (SEQ ID NO: 186) | |
| Primer Pair 1 | Reverse | AAACCAACACCATCCTCAAACT (SEQ ID | 130 |
| | Primer | NO: 187) | |
| | Product | | |
| Primer Pair 2 | Forward | AGGGTGAGTTAATTGTTTATTGTTGTTT | |
| | Primer | (SEQ ID NO: 188) | |
| Primer Pair 2 | Reverse | ACATACCCACCCTCTAATATATCTCAA | 250 |
| | Primer | (SEQ ID NO: 189) | |
| | Product | | |
| Primer Pair 3 | Forward | GGGTAGGAGGTTGTTATTT (SEQ ID NO: | |
| | Primer | 190) | |
| Primer Pair 3 | Reverse | ACATACCCACCCTCTAATA (SEQ ID NO: | 165 |
| | Primer | 191) | |
| | Product | | |
| Primer Pair 4 | Forward | TTAGTATGGGTAGAAGGG (SEQ ID NO: | |
| | Primer | 192) | |
| Primer Pair 4 | Reverse | AAATTCTAACTAAACCACAAA (SEQ ID NO: | 124 |
| | Primer | 193) | |
| | Product | | |
| | Type | Sequence23 | Amplicon |
| | Probe- | TGAGGATGGTGTTG (SEQ ID NO: 194) | |
| | Unmeth | | |
| | Probe_meth | TGAGGACGGTGTTG (SEQ ID NO: 195) | |
| Primer Pair 1 | Forward | AGGGTGAGTTAATTGTTTATTGTTGTTT | |
| | Primer | (SEQ ID NO: 196) | |
| Primer Pair 1 | Reverse | ACATACCCACCCTCTAATATATCTCAA | 250 |
| | Primer | (SEQ ID NO: 197) | |
| | Product | | |
| Primer Pair 2 | Forward | GGGTAGGAGGTTGTTATTTAGT (SEQ ID | |
| | Primer | NO: 198) | |
| Primer Pair 2 | Reverse | AAACATACCCACCCTCTAATA (SEQ ID NO: | 167 |
| | Primer | 199) | |
| | Product | | |
| Primer Pair 3 | Forward | TTTGTGGTTTAGTTAGAATTT (SEQ ID NO: | |
| | Primer | 200) | |
| Primer Pair 3 | Reverse | CCCTCCTCCAAACATAA (SEQ ID NO: 201) | 250 |
| | Primer | | |
| | Product | | |
| | Type | Sequence24 | Amplicon |
| | Probe- | TGGTTTTGGTAGTT (SEQ ID NO: 201) | |
| | Unmeth | | |
| | Probe_meth | TGGTTTCGGTAGTT (SEQ ID NO: 201) | |
| Primer Pair 1 | Forward | AGGGTGAGTTAATTGTTTATTGTTGTTT | |
| | Primer | (SEQ ID NO: 202) | |
| Primer Pair 1 | Reverse | ACATACCCACCCTCTAATATATCTCAA | 250 |
| | Primer | (SEQ ID NO: 203) | |
| | Product | | |
| Primer Pair 2 | Forward | GGGTAGGAGGTTGTTATTTAGT (SEQ ID | |
| | Primer | NO: 204) | |
| Primer Pair 2 | Reverse | AAACATACCCACCCTCTAATA (SEQ ID NO: | 167 |
| | Primer | 205) | |
| | Product | | |
| Primer Pair 3 | Forward | TTAGTTTGAGGATGGTGT (SEQ ID NO: | |
| | Primer | 206) | |
| Primer Pair 3 | Reverse | CCTCCTCCAAACATAATAAA (SEQ ID NO: | 229 |
| | Primer | 207) | |
| | Product | | |
| Primer Pair 4 | Forward | TTTGTGGTTTAGTTAGAATTT (SEQ ID NO: | |
| | Primer | 208) | |
| Primer Pair 4 | Reverse | AAACACTTAAATCTACAATCAT (SEQ ID | 161 |
| | Primer | NO: 209) | |
| | Product | | |
| | Type | Sequence25 | Amplicon |
| | Probe- | AGTTTTGAGATA (SEQ ID NO: 210) | |
| | Unmeth | | |
| | Probe_meth | AGTTTCGAGATA (SEQ ID NO: 211) | |
| Primer Pair 1 | Forward | TTGAGGATGGTGTTGGTTT (SEQ ID NO: | |
| | Primer | 212) | |
| Primer Pair 1 | Reverse | CACCCTCCTCCAAACATAATAA (SEQ ID | 227 |
| | Primer | NO: 213) | |
| | Product | | |
| Primer Pair 2 | Forward | GGGTAGGAGGTTGTTATTTAGT (SEQ ID | |
| | Primer | NO: 214) | |
| Primer Pair 2 | Reverse | AAACATACCCACCCTCTAATA (SEQ ID NO: | 167 |
| | Primer | 215) | |
| Primer Pair 3 | Product | | |
| | Forward | TTTAGTTTGAGGATGGTG (SEQ ID NO: | |
| | Primer | 216) | |
| Primer Pair 3 | Reverse | AAACACTTAAATCTACAATCAT (SEQ ID | 142 |
| | Primer | NO: 217) | |
| | Product | | |
| Primer Pair 4 | Forward | TGTGGTTTAGTTAGAATTT (SEQ ID NO: | |
| | Primer | 218) | |
| Primer Pair 4 | Reverse | AATCTACAATCATCAAATAA (SEQ ID NO: | 150 |
| | Primer | 219) | |
| | Product | | |
| | Type | Sequence26 | Amplicon |
| | Probe- | TGGGTATGTTTTT (SEQ ID NO: 220) | |
| | Unmeth | | |
| | Probe_meth | TGGGTACGTTTTT (SEQ ID NO: 221) | |
| Primer Pair 1 | Forward | TTGAGGATGGTGTTGGTTT (SEQ ID NO: | |
| | Primer | 222) | |
| Primer Pair 1 | Reverse | CACCCTCCTCCAAACATAATAA (SEQ ID | 227 |
| | Primer | NO: 223) | |
| | Product | | |
| Primer Pair 2 | Forward | GGGTAGGAGGTTGTTATTTAGTAGG (SEQ | |
| | Primer | ID NO: 224) | |
| Primer Pair 2 | Reverse | AAACACTTAAATCTACAATCATCAAATAAA | 247 |
| | Primer | (SEQ ID NO: 225) | |
| | Product | | |
| Primer Pair 3 | Forward | GGGTAGGAGGTTGTTATT (SEQ ID NO: | |
| | Primer | 226) | |
| Primer Pair 3 | Reverse | AAACACTTAAATCTACAATCAT (SEQ ID | 247 |
| | Primer | NO: 227) | |
| | Product | | |
| Primer Pair 4 | Forward | TTTAGTTTGAGGATGGTG (SEQ ID NO: | |
| | Primer | 228) | |
| Primer Pair 4 | Reverse | CAAACAAACAACCACAC (SEQ ID NO: 229) | 247 |
| | Primer | | |
| | Product | | |
| | Type | Sequence27 | Amplicon |
| | Probe- | TTATTTGTTTTT (SEQ ID NO: 230) | |
| | Unmeth | | |
| | Probe_meth | TTATTCGTTTTT (SEQ ID NO: 231) | |
| Primer Pair 1 | Forward | TTTGAGATATATTAGAGGGTGGGTATGT | |
| | Primer | (SEQ ID NO: 232) | |
| Primer Pair 1 | Reverse | AACCCACTCAAACAAACAACCA (SEQ ID | 223 |
| | Primer | NO: 233) | |
| | Product | | |
| Primer Pair 2 | Forward | TTGAGGATGGTGTTGGTTT (SEQ ID NO: | |
| | Primer | 234) | |
| Primer Pair 2 | Reverse | CACCCTCCTCCAAACATAATAA (SEQ ID | 227 |
| | Primer | NO: 235) | |
| | Product | | |
| Primer Pair 3 | Forward | GGGTAGGAGGTTGTTATTTAGTAGG (SEQ | |
| | Primer | ID NO: 236) | |
| Primer Pair 3 | Reverse | AAACACTTAAATCTACAATCATCAAATAAA | 247 |
| | Primer | (SEQ ID NO: 237) | |
| | Product | | |
| Primer Pair 4 | Forward | GGGTAGGAGGTTGTTATT (SEQ ID NO: | |
| | Primer | 238) | |
| Primer Pair 4 | Reverse | AAACACTTAAATCTACAATCAT (SEQ ID | 247 |
| | Primer | NO: 239) | |
| | Product | | |
| | Type | Sequence28 | Amplicon |
| | Probe- | TGTTTTGTAGTT (SEQ ID NO: 240) | |
| | Unmeth | | |
| | Probe_meth | TGTTTCGTAGTT (SEQ ID NO: 241) | |
| Primer Pair 1 | Forward | GAGATATATTAGAGGGTGGGTATGTT | |
| | Primer | (SEQ ID NO: 242) | |
| Primer Pair 1 | Reverse | ACACCCTCCTCCAAACATAATAAA (SEQ ID | 199 |
| | Primer | NO: 243) | |
| | Product | | |
| Primer Pair 2 | Forward | GGGATAGTAGTGTAAAGAGTTT (SEQ ID | |
| | Primer | NO: 244) | |
| Primer Pair 2 | Reverse | CCTATCCCTACTCACAACTAAA (SEQ ID | 124 |
| | Primer | NO: 245) | |
| | Product | | |
| Primer Pair 3 | Forward | TGAGGATGGTGTTGGTT (SEQ ID NO: 246) | |
| | Primer | | |
| Primer Pair 3 | Reverse | CACTTAAATCTACAATCATCAAATAAA | 132 |
| | Primer | (SEQ ID NO: 247) | |
| | Product | | |
| Primer Pair 4 | Forward | TTTGTGGTTTAGTTAGAATTT (SEQ ID NO: | |
| | Primer | 24\8) | |
| Primer Pair 4 | Reverse | AAACACTTAAATCTACAATCAT (SEQ ID | 161 |
| | Primer | NO: 249) | |
| | Product | | |
| | Type | Sequence29 | Amplicon |
| | Probe- | ATGATTGTAGA (SEQ ID NO: 250) | |
| | Unmeth | | |
| | Probe_meth | ATGATCGTAGA (SEQ ID NO: 251) | |
| Primer Pair 1 | Forward | GAGATATATTAGAGGGTGGGTATGTT | |
| | Primer | (SEQ ID NO: 252) | |
| Primer Pair 1 | Reverse | ACACCCTCCTCCAAACATAATAAA (SEQ ID | 199 |
| | Primer | NO: 253) | |
| | Product | | |
| | Type | Sequence30 | Amplicon |
| | Probe- | TGTTTTATGTTGTTTGT (SEQ ID NO: 254) | |
| | Unmeth | | |
| | Probe_meth | TGTTTTACGTTGTTTGT (SEQ ID NO: 255) | |
| Primer Pair 1 | Forward | GAGATATATTAGAGGGTGGGTATGTT | |
| | Primer | (SEQ ID NO: 256) | |
| Primer Pair 1 | Reverse | ACACCCTCCTCCAAACATAATAAA (SEQ ID | 199 |
| | Primer | NO: 257) | |
| | Product | | |
| Primer Pair 2 | Forward | TTTATTTGATGATTGTAGATTTAAGTGTTT | |
| | Primer | (SEQ ID NO: 258) | |
| Primer Pair 2 | Reverse | AAACAACCACACCCTCCT (SEQ ID NO: | 130 |
| | Primer | 259) | |
| | Product | | |
| Primer Pair 3 | Forward | TATTTGATGATTGTAGATTT (SEQ ID NO: | |
| | Primer | 260) | |
| Primer Pair 3 | Reverse | CCCATCTCCTAACTATAA (SEQ ID NO: 261) | 189 |
| | Primer | | |
| | Product | | |
| | Type | Sequence31 | Amplicon |
| | Probe- | TTGGGTGAATAT (SEQ ID NO: 262) | |
| | Unmeth | | |
| | Probe_meth | TTGGGCGAATAT (SEQ ID NO: 263) | |
| Primer Pair 1 | Forward | GAGATATATTAGAGGGTGGGTATGTT | |
| | Primer | (SEQ ID NO: 264) | |
| Primer Pair 1 | Reverse | ACACCCTCCTCCAAACATAATAAA (SEQ ID | 199 |
| | Primer | NO: 265) | |
| | Product | | |
| Primer Pair 2 | Forward | TTTATTTGATGATTGTAGATTTAAGTGTTT | |
| | Primer | (SEQ ID NO: 266) | |
| Primer Pair 2 | Reverse | AAACAACCACACCCTCCT (SEQ ID NO: | 130 |
| | Primer | 267) | |
| | Product | | |
| Primer Pair 3 | Forward | TATTTGATGATTGTAGATTT (SEQ ID NO: | |
| | Primer | 268) | |
| Primer Pair 3 | Reverse | CCCATCTCCTAACTATAA (SEQ ID NO: 269) | 189 |
| | Primer | | |
| | Product | | |
| | Type | Sequence32 | Amplicon |
| | Probe- | ATTATGTTTGGAGG (SEQ ID NO: 270) | |
| | Unmeth | | |
| | Probe_meth | ATTACGTTCGGAGG (SEQ ID NO: 271) | |
| Primer Pair 1 | Forward | TTTATTTGATGATTGTAGATTTAAGTGTTT | |
| | Primer | (SEQ ID NO: 272) | |
| Primer Pair 1 | Reverse | CAAACAAACAACCACACCCT (SEQ ID NO: | 135 |
| | Primer | 273) | |
| | Product | | |
| Primer Pair 2 | Forward | TATTTGATGATTGTAGATTT (SEQ ID NO: | |
| | Primer | 274) | |
| Primer Pair 2 | Reverse | CCCATCTCCTAACTATAA (SEQ ID NO: 275) | 189 |
| | Primer | | |
| | Product | | |
| | Type | Sequence33 | Amplicon |
| | Probe- | AGGAGGGTGTGGTTG (SEQ ID NO: 276) | |
| | Unmeth | | |
| | Probe_meth | AGGAGGGCGTGGTTG (SEQ ID NO: 277) | |
| Primer Pair 1 | Forward | TTGATGATTGTAGATTTAAGTGTTT (SEQ | |
| | Primer | ID NO: 278) | |
| Primer Pair 1 | Reverse | AAATCTAACCCACTCAAACAAA (SEQ ID | 144 |
| | Primer | NO: 279) | |
| | Product | | |
| Primer Pair 2 | Forward | TATTTGATGATTGTAGATTT (SEQ ID NO: | |
| | Primer | 280) | |
| Primer Pair 2 | Reverse | CCCATCTCCTAACTATAA (SEQ ID NO: 281) | 189 |
| | Primer | | |
| | Product | | |
| | Type | Sequence34 | Amplicon |
| | Probe- | TTTTGTTGTTAGG (SEQ ID NO: 282) | |
| | Unmeth | | |
| | Probe_meth | TTTTGTCGTTAGG (SEQ ID NO: 283) | |
| Primer Pair 1 | Forward | TGGTTGTTTGTTTGAGTGGGTTAGA (SEQ | |
| | Primer | ID NO: 284) | |
| Primer Pair 1 | Reverse | ACCCACATATCCCACCTCCTT (SEQ ID NO: | 174 |
| | Primer | 285) | |
| | Product | | |
| Primer Pair 2 | Forward | TTTGGAGGAGGGTGTGGTTG (SEQ ID NO: | |
| | Primer | 286) | |
| Primer Pair 2 | Reverse | AACCTAAACAAATAATCCCAATACTTCTCC | 138 |
| | Primer | (SEQ ID NO: 287) | |
| | Product | | |
| Primer Pair 3 | Forward | GAGGGTGTGGTTGTTTGTTTGA (SEQ ID | |
| | Primer | NO: 288) | |
| Primer Pair 3 | Reverse | CCCAACACCCACATATCCCA (SEQ ID NO: | 187 |
| | Primer | 289) | |
| | Product | | |
| Primer Pair 4 | Forward | TTATGTTTGGAGGAGGGT (SEQ ID NO: | |
| | Primer | 290) | |
| Primer Pair 4 | Reverse | AAACCTAAACAAATAATCCCAATA (SEQ ID | 144 |
| | Primer | NO: 291) | |
| | Product | | |
| Primer Pair 5 | Forward | TGATTGTAGATTTAAGTGTTT (SEQ ID NO: | |
| | Primer | 292) | |
| Primer Pair 5 | Reverse | CCCATCTCCTAACTATAAA (SEQ ID NO: | 182 |
| | Primer | 293) | |
| | Product | | |
| | Type | Sequence35 | Amplicon |
| | Probe- | TTTTATGGTAG (SEQ ID NO: 294) | |
| | Unmeth | | |
| | Probe_meth | TTTTACGGTAG (SEQ ID NO: 295) | |
| Primer Pair 1 | Forward | TGGTTGTTTGTTTGAGTGGGTTAGA (SEQ | |
| | Primer | ID NO: 296) | |
| Primer Pair 1 | Reverse | ACCCACATATCCCACCTCCTT (SEQ ID NO: | 174 |
| | Primer | 297) | |
| | Product | | |
| Primer Pair 2 | Forward | TTTGGAGGAGGGTGTGGTTG (SEQ ID NO: | |
| | Primer | 298) | |
| Primer Pair 2 | Reverse | AACCTAAACAAATAATCCCAATACTTCTCC | 138 |
| | Primer | (SEQ ID NO: 299) | |
| | Product | | |
| Primer Pair 3 | Forward | GAGGGTGTGGTTGTTTGTTTGA (SEQ ID | |
| | Primer | NO: 300) | |
| Primer Pair 3 | Reverse | CCCAACACCCACATATCCCA (SEQ ID NO: | 187 |
| | Primer | 301) | |
| | Product | | |
| Primer Pair 4 | Forward | TTATGTTTGGAGGAGGGT (SEQ ID NO: | |
| | Primer | 302) | |
| Primer Pair 4 | Reverse | AAACCTAAACAAATAATCCCAATA (SEQ ID | 144 |
| | Primer | NO: 303) | |
| | Product | | |
| Primer Pair 5 | Forward | TGATTGTAGATTTAAGTGTTT (SEQ ID NO: | |
| | Primer | 304) | |
| Primer Pair 5 | Reverse | CCCATCTCCTAACTATAAA (SEQ ID NO: | 182 |
| | Primer | 305) | |
| | Product | | |
| | Type | Sequence36 | Amplicon |
| | Probe- | TGTGGGTGTTGGG (SEQ ID NO: 306) | |
| | Unmeth | | |
| | Probe_meth | TGTGGGCGTTGGG (SEQ ID NO: 307) | |
| Primer Pair 1 | Forward | GGGAGAAGTATTGGGATTATTTGTTTAGGT | |
| | Primer | (SEQ ID NO: 308) | |
| Primer Pair 1 | Reverse | ACACTCCCACCCATCTCCAA (SEQ ID NO: | 164 |
| | Primer | 309) | |
| | Product | | |
| Primer Pair 2 | Forward | GGGAAGGAGGTGGGATA (SEQ ID NO: | |
| | Primer | 310) | |
| Primer Pair 2 | Reverse | CCATCTCCAACCAAACTAAAC (SEQ ID NO: | 97 |
| | Primer | 311) | |
| | Product | | |
| Primer Pair 3 | Forward | GGGAGAAGTATTGGGATTATTTG (SEQ ID | |
| | Primer | NO: 312) | |
| Primer Pair 3 | Reverse | CTACCCACCAACCCTAAATC (SEQ ID NO: | 184 |
| | Primer | 313) | |
| | Product | | |
| Primer Pair 4 | Forward | TTTGTTTGAGTGGGTTAGA (SEQ ID NO: | |
| | Primer | 314) | |
| Primer Pair 4 | Reverse | CCACACTAAATATAAACCTACAA (SEQ ID | 199 |
| | Primer | NO: 315) | |
| | Product | | |
| Primer Pair 5 | Forward | TTTATAGTTAGGAGATGGG (SEQ ID NO: | |
| | Primer | 316) | |
| Primer Pair 5 | Reverse | CCAAACTAAACCCAAATTA (SEQ ID NO: | 186 |
| | Primer | 317) | |
| | Product | | |
| | Type | Sequence37 | Amplicon |
| | Probe- | TTAGTTTGGTTGG (SEQ ID NO: 318) | |
| | Unmeth | | |
| | Probe_meth | TTAGTTCGGTTGG (SEQ ID NO: 319) | |
| Primer Pair 1 | Forward | GGAGGTGGGATATGTGGGTGT (SEQ ID | |
| | Primer | NO: 320) | |
| Primer Pair 1 | Reverse | ACCTACCCACCAACCCTAAATCA (SEQ ID | 124 |
| | Primer | NO: 321) | |
| | Product | | |
| Primer Pair 2 | Forward | GGGAGAAGTATTGGGATTATTTGTTTAGGT | |
| | Primer | (SEQ ID NO: 322) | |
| Primer Pair 2 | Reverse | ACAATACCCACCTACCCACCAA (SEQ ID | 195 |
| | Primer | NO: 323) | |
| | Product | | |
| Primer Pair 3 | Forward | GTTTGTAGGTTTATATTTAGTGTGGGTGAT | |
| | Primer | (SEQ ID NO: 324) | |
| Primer Pair 3 | Reverse | CTAAATCACACTCCCACCCATCT (SEQ ID | 84 |
| | Primer | NO: 325) | |
| | Product | | |
| Primer Pair 4 | Forward | GGGAAGGAGGTGGGATA (SEQ ID NO: | |
| | Primer | 326) | |
| Primer Pair 4 | Reverse | ACCCTAAATCACACTCCCA (SEQ ID NO: | 117 |
| | Primer | 327) | |
| | Product | | |
| Primer Pair 5 | Forward | GGGAGAAGTATTGGGATTA (SEQ ID NO: | |
| | Primer | 328) | |
| Primer Pair 5 | Reverse | AAACACAATACCCACCTA (SEQ ID NO: | 199 |
| | Primer | 329) | |
| | Product | | |
| | Type | Sequence38 | Amplicon |
| | Probe- | AGTGTGATTTA (SEQ ID NO: 330) | |
| | Unmeth | | |
| | Probe_meth | AGTGCGATTTA (SEQ ID NO: 331) | |
| Primer Pair 1 | Forward | GGGAGAAGTATTGGGATTATTTGTTTAGGT | |
| | Primer | (SEQ ID NO: 332) | |
| Primer Pair 1 | Reverse | ACAATACCCACCTACCCACCAA (SEQ ID | 195 |
| | Primer | NO: 333) | |
| | Product | | |
| Primer Pair 2 | Forward | AATTTGGGTTTAGTTTGGTTGGAGA (SEQ | |
| | Primer | ID NO: 334) | |
| Primer Pair 2 | Probe | AGTGTGATTTA (SEQ ID NO: 335) | |
| | Reverse | CACCCAACTCCACCTACCC (SEQ ID NO: | 169 |
| Primer Pair 3 | Primer | 336) | |
| | Product | | |
| Primer Pair 3 | Forward | TTTAATTTGGGTTTAGTTTGG (SEQ ID NO: | |
| | Primer | 337) | |
| Primer Pair 4 | Probe | AGTGTGATTTA (SEQ ID NO: 338) | |
| | Reverse | AACACAATACCCACCTAC (SEQ ID NO: | 75 |
| Primer Pair 4 | Primer | 339) | |
| | Product | | |
| | Type | Sequence39 | Amplicon |
| | Probe- | TTGGCGGGTAG (SEQ ID NO: 340) | |
| | Unmeth | | |
| | Probe_meth | TTGGCGGGTAG (SEQ ID NO: 341) | |
| Primer Pair 1 | Forward | GAGATGGGTGGGAGTGTGATTTA (SEQ ID | |
| | Primer | NO: 342) | |
| Primer Pair 1 | Reverse | CACCCAACTCCACCTACCC (SEQ ID NO: | 148 |
| | Primer | 343) | |
| | Product | | |
| Primer Pair 2 | Forward | TTGGAGATGGGTGGGAGTG (SEQ ID NO: | |
| | Primer | 344) | |
| Primer Pair 2 | Reverse | AAACTACAAACTACCTACACCAAA(SEQ ID | 180 |
| | Primer | NO: 345) | |
| | Product | | |
| | Type | Sequence40 | Amplicon |
| | Probe- | AGGTGGGTA (SEQ ID NO: 346) | |
| | Unmeth | | |
| | Probe_meth | AGGCGGGTA (SEQ ID NO: 347) | |
| Primer Pair 1 | Forward | GAGATGGGTGGGAGTGTGATTTA (SEQ ID | |
| | Primer | NO: 348) | |
| Primer Pair 1 | Reverse | CACCCAACTCCACCTACCC (SEQ ID NO: | 148 |
| | Primer | 349) | |
| | Product | | |
| Primer Pair 2 | Forward | TTGGAGATGGGTGGGAGTG (SEQ ID NO: | |
| | Primer | 350) | |
| Primer Pair 2 | Reverse | AAACTACAAACTACCTACACCAAA (SEQ ID | 180 |
| | Primer | NO: 351) | |
| | Product | | |
| Primer Pair 3 | Forward | GATTTAGGGTTGGTGGGT (SEQ ID NO: | |
| | Primer | 352) | |
| Primer Pair 3 | Reverse | CCACAATACCACACTTCTACA (SEQ ID NO: | 200 |
| | Primer | 353) | |
| | Product | | |
| | Type | Sequence41 | Amplicon |
| | Probe- | TGTTTTGTTGTTGTT (SEQ ID NO: 354) | |
| | Unmeth | | |
| | Probe_meth | TGTTTCGTCGTTGTT (SEQ ID NO: 355) | |
| Primer Pair 1 | Forward | GAGATGGGTGGGAGTGTGATTTA (SEQ ID | |
| | Primer | NO: 356) | |
| Primer Pair 1 | Reverse | CACCCAACTCCACCTACCC(SEQ ID NO: | 148 |
| | Primer | 357) | |
| | Product | | |
| Primer Pair 2 | Forward | TTTAGGGTTGGTGGGTAGGT (SEQ ID NO: | |
| | Primer | 358) | |
| Primer Pair 2 | Reverse | TTCCACAATACCACACTTCTACAAA (SEQ | 200 |
| | Primer | ID NO: 359) | |
| | Product | | |
| Primer Pair 3 | Forward | TTGGAGATGGGTGGGAGTG (SEQ ID NO: | |
| | Primer | 360) | |
| Primer Pair 3 | Reverse | AAACTACAAACTACCTACACCAAA (SEQ ID | 180 |
| | Primer | NO: 361) | |
| | Product | | |
| Primer Pair 4 | Forward | GTAGGTGGGTATTGTGTTT (SEQ ID NO: | |
| | Primer | 362) | |
| Primer Pair 4 | Reverse | CTAATACAACATTATTCCACAAT (SEQ ID | 200 |
| | Primer | NO: 363) | |
| | Product | | |
| | Type | Sequence42 | Amplicon |
| | Probe- | TGTTTTGGAAT (SEQ ID NO: 364) | |
| | Unmeth | | |
| | Probe_meth | TGTTTCGGAAT (SEQ ID NO: 365) | |
| Primer Pair 1 | Forward | GAGATGGGTGGGAGTGTGATTTA (SEQ ID | |
| | Primer | NO: 366) | |
| Primer Pair 1 | Reverse | CACCCAACTCCACCTACCC (SEQ ID NO: | 148 |
| | Primer | 367) | |
| | Product | | |
| Primer Pair 2 | Forward | TTTAGGGTTGGTGGGTAGGT (SEQ ID NO: | |
| | Primer | 368) | |
| Primer Pair 2 | Reverse | TTCCACAATACCACACTTCTACAAA (SEQ | 200 |
| | Primer | ID NO: 369) | |
| | Product | | |
| Primer Pair 3 | Forward | TTGGAGATGGGTGGGAGTG (SEQ ID NO: | |
| | Primer | 370) | |
| Primer Pair 3 | Reverse | AAACTACAAACTACCTACACCAAA (SEQ ID | 180 |
| | Primer | NO: 371) | |
| | Product | | |
| Primer Pair 4 | Forward | GTAGGTGGGTATTGTGTTT (SEQ ID NO: | |
| | Primer | 372) | |
| Primer Pair 4 | Reverse | CTAATACAACATTATTCCACAAT (SEQ ID | 200 |
| | Primer | NO: 373) | |
| | Product | | |
| | Type | Sequence43 | Amplicon |
| | Probe- | TTTTGTGTGGTATGTTTT (SEQ ID NO: 374) | |
| | Unmeth | | |
| | Probe_meth | TTTTGCGCGGTATGTTTT (SEQ ID NO: 375) | |
| Primer Pair 1 | Forward | GAGATGGGTGGGAGTGTGATTTA (SEQ ID | |
| | Primer | NO: 376) | |
| Primer Pair 1 | Reverse | CACCCAACTCCACCTACCC (SEQ ID NO: | 148 |
| | Primer | 377) | |
| | Product | | |
| Primer Pair 2 | Forward | TTTAGGGTTGGTGGGTAGGT (SEQ ID NO: | |
| | Primer | 378) | |
| Primer Pair 2 | Reverse | TTCCACAATACCACACTTCTACAAA (SEQ | 200 |
| | Primer | ID NO: 379) | |
| | Product | | |
| Primer Pair 3 | Forward | TTGGAGATGGGTGGGAGTG (SEQ ID NO: | |
| | Primer | 380) | |
| Primer Pair 3 | Reverse | AAACTACAAACTACCTACACCAAA (SEQ ID | 180 |
| | Primer | NO: 381) | |
| | Product | | |
| Primer Pair 4 | Forward | GTAGGTGGGTATTGTGTTT (SEQ ID NO: | |
| | Primer | 382) | |
| Primer Pair 4 | Reverse | CTAATACAACATTATTCCACAAT (SEQ ID | 200 |
| | Primer | NO: 383) | |
| | Product | | |
| | Type | Sequence45 | Amplicon |
| | Probe- | TTGGGTGGGGGT (SEQ ID NO: 384) | |
| | Unmeth | | |
| | Probe_meth | TTGGGCGGGGGT (SEQ ID NO: 385) | |
| Primer Pair 1 | Forward | TTTAGGGTTGGTGGGTAGGT (SEQ ID NO: | |
| | Primer | 386) | |
| Primer Pair 1 | Reverse | TTCCACAATACCACACTTCTACAAA (SEQ | 200 |
| | Primer | ID NO: 387) | |
| | Product | | |
| Primer Pair 2 | Forward | GATGGGTGGGAGTGTGATT (SEQ ID NO: | |
| | Primer | 388) | |
| Primer Pair 2 | Reverse | AAACTACAAACTACCTACACCAAA (SEQ ID | 175 |
| | Primer | NO: 389) | |
| | Product | | |
| Primer Pair 3 | Forward | GTAGGTGGGTATTGTGTTT (SEQ ID NO: | |
| | Primer | 390) | |
| Primer Pair 3 | Reverse | CTAATACAACATTATTCCACAAT (SEQ ID | 200 |
| | Primer | NO: 391) | |
| | Product | | |
| | Type | Sequence46 | Amplicon |
| | Probe- | TAGAAGTGTGGTA (SEQ ID NO: 392) | |
| | Unmeth | | |
| | Probe_meth | TAGAAGCGTGGTA (SEQ ID NO: 393) | |
| Primer Pair 1 | Forward | TTTGGTGTAGGTAGTTTGTAGTTT (SEQ ID | |
| | Primer | NO: 394) | |
| Primer Pair 1 | Reverse | AACTTTATTCCATCTCTCTCAATACA (SEQ | 182 |
| | Primer | ID NO: 395) | |
| | Product | | |
| Primer Pair 2 | Forward | GGGTAGGTGGAGTTGGGT(SEQ ID NO: | |
| | Primer | 396) | |
| Primer Pair 2 | Reverse | AACAAATACTAATACAACATTATTCCACAA | 112 |
| | Primer | (SEQ ID NO: 397) | |
| | Product | | |
| Primer Pair 3 | Forward | TTTGGTGTAGGTAGTTT (SEQ ID NO: 398) | |
| | Primer | | |
| Primer Pair 3 | Reverse | CAATAATTCTCCAACTAATAAA (SEQ ID | 113 |
| | Primer | NO: 399) | |
| | Product | | |
| | Type | Sequence47 | Amplicon |
| | Probe- | ATTAGATGTAGTT (SEQ ID NO: 400) | |
| | Unmeth | | |
| | Probe_meth | ATTAGACGTAGTT (SEQ ID NO: 401) | |
| Primer Pair 1 | Forward | TGTAGAAGTGTGGTATTGTGGAATAAT | |
| | Primer | (SEQ ID NO: 402) | |
| Primer Pair 1 | Reverse | AAACTTTATTCCATCTCTCTCAATACAAA | 140 |
| | Primer | (SEQ ID NO: 403) | |
| | Product | | |
| Primer Pair 2 | Forward | TTTGTAGAAGTGTGGTATTGT (SEQ ID NO: | |
| | Primer | 404) | |
| Primer Pair 2 | Reverse | AAACTTTATTCCATCTCTCTCA (SEQ ID | 142 |
| | Primer | NO: 405) | |
| | Product | | |
| | Type | Sequence48 | Amplicon |
| | Probe- | TAGTTTGTAGG (SEQ ID NO: 406) | |
| | Unmeth | | |
| | Probe_meth | TAGTTCGTAGG (SEQ ID NO: 407) | |
| Primer Pair 1 | Forward | TGTAGAAGTGTGGTATTGTGGAATAAT | |
| | Primer | (SEQ ID NO: 408) | |
| Primer Pair 1 | Reverse | AAACTTTATTCCATCTCTCTCAATACAAA | 140 |
| | Primer | (SEQ ID NO: 409) | |
| | Product | | |
| Primer Pair 2 | Forward | TTTGTAGAAGTGTGGTATTGT (SEQ ID NO: | |
| | Primer | 410) | |
| Primer Pair 2 | Reverse | AAACTTTATTCCATCTCTCTCA (SEQ ID | 142 |
| | Primer | NO: 411) | |
| | Product | | |
| | Type | Sequence49 | Amplicon |
| | Probe- | ATTTGTTGTTTT (SEQ ID NO: 412) | |
| | Unmeth | | |
| | Probe_meth | ATTCGTCGTTTT (SEQ ID NO: 413) | |
| Primer Pair 1 | Forward | GTAGAAGTGTGGTATTGTGGAATAAT (SEQ | |
| | Primer | ID NO: 414) | |
| Primer Pair 1 | Reverse | AAACTTTATTCCATCTCTCTCAATACA | 139 |
| | Primer | (SEQ ID NO: 415) | |
| | Product | | |
| Primer Pair 2 | Forward | TTGTAGAAGTGTGGTATTG (SEQ ID NO: | |
| | Primer | 416) | |
| Primer Pair 2 | Reverse | AAACTTTATTCCATCTCTCT (SEQ ID NO: | 141 |
| | Primer | 417) | |
| | Product | | |

### Exemplification

The invention now being generally described, will be more readily understood by reference to the following example, which is included merely for purposes of illustration and is not intended to be limiting.

### Experimental Design

All CpG sites for the human insulin gene (hg19_knownGene_uc021qcd.1 range=chr11:2181009-2182439) were mapped. The sequence was then transformed to a sequence representing bisulfite-treated DNA, where all Cs are converted to Ts and in the case of a methylated CpG site, the C is protected from conversion to T. The probes were designed to include two CpG sites at nucleotides 21814010 and 21814012 (http://genome.ucsc.edu/cgi-bin/hgGateway, Feb 2009 GRCh37/hgl9) in positions +396 and +399 from the transcription start site.

The nucloetide sequence of the methylation sensi-
tive probe is:
(SEQ ID NO: 1)
ATTTAAGATTTGTTGGGAGGTAGAG
and
the nucleotide sequence of the methylation
insensitive probe is:
(SEQ ID NO: 2)
ATTTAAGATTCGTCGGGAGGTAGAG.
The sequences of the forward and reverse primers
are:
(SEQ ID NO: 5)
GTGGTTTATATTTGGTGGA
and
(SEQ ID NO: 6)
ATTAACTCACCCTACAAATC.

To increase sensitivity, specificity and to reduce background fluorescence, the probes were designed with an internal Zen quencher (ZEN) in addition to the 3′ Iowa Black FQ quencher (3IABkFQ). The ZEN probes were synthesized by Integrated DNA Technologies.

The synthetic L2_M and L2_UM sequences, which are replicas of bisulfite-treated methylated and un-methylated human insulin gene sequences (region 2181155-2181465 on Chr11), respectively, were cloned into pUC57 plasmids (Genewiz, Inc).

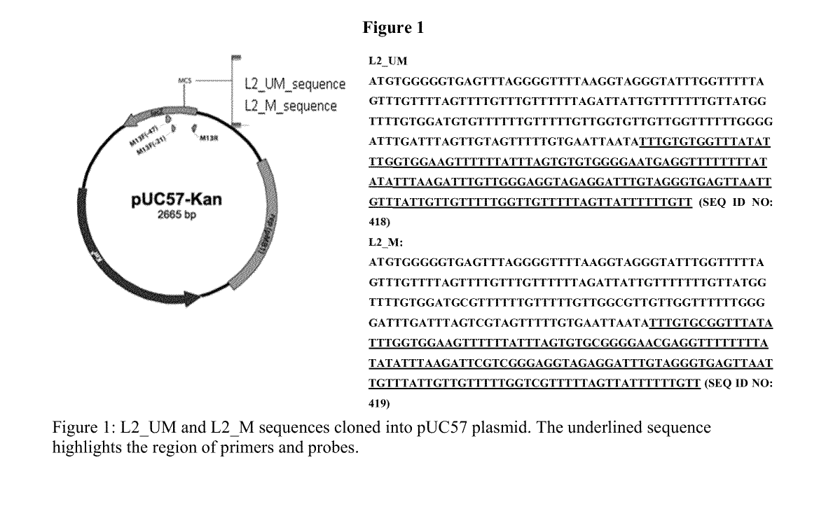

FIG. 1 illustrates the cloned sequences in pUC57 Kan plasmid. The plasmids were used for optimization of PCR conditions and to determine the sensitivity and specificity of the primers and probes.

Reaction mixtures of 25 ul volume, comprising 1× Droplet PCR supermix, (BioRad), 900 uM primer mix, 250 uM probe mix, 5 ul of plasmid (copy number ranging from 100,000 to 1 and mixed populations of un-methylated and methylated plasmids) or 4 ul of bisulfite treated genomic DNA from either serum, islet cells or peripheral blood mononuclear cells (PBMCs) was prepared. Twenty μl of the PCR reaction mix was loaded into a separate well of a eight channel droplet generator cartridge, and in a separate corresponding well 70 ul of droplet generation oil (BioRad) was loaded. The cartridge was loaded into a droplet generator (Biorad). Forty ul of the generated droplets were carefully transferred to a 96 well PCR plate and the PCR reaction was run on a thermal cycle with the following protocol: 10 min activation at 95° C. followed by 40 cycles of a two-step amplification protocol of 30 seconds at 94° C. denaturation and 60 seconds at 58° C. for a combined annealing-extension step. A final 10 min at 98° C. inactivation step completed the reaction.

The PCR plate was then transferred to a QX100 droplet reader (Biorad), which automatically reads the droplets from each well of the plate. Analysis of ddPCR data was performed using QuantaSoft Analysis software.

Islet, PBMCs and serum samples from non-diabetic and diabetic patients were used to validate the protocol developed using the plasmids. DNA was isolated using the Qiagen blood and tissue DNA extraction kit. DNA was bisulfite treated using an EZ DNA Methylation kit (Zymo Research, Irvine, Calif.).

Limit of detection assays were performed with plasmids, islets and PBMCs. The plasmid suspensions were made in a series of 10 fold serial dilutions from 100,000 copies to 1 copy. Bisulfite-treated DNA from islets and PBMCs were diluted over a ¼dilution series up to 1/1024 with amounts ranging from 148 ng to 0.1 ng.

### Results

In the multiplexed assay, the probe for unmethylated human insulin gene DNA successfully detected 1 copy/μl of plasmid and showed no cross amplification with the plasmid representing methylated DNA. The probe for methylated DNA successfully detected 1 copy/ul per reaction and showed minimal cross amplification only with high numbers of plasmid representing un-methylated insulin gene DNA.

In the recovery assay, where approximately 3000 copies/ul of L2_M plasmid were mixed with 10 copies of the L2_UM plasmid, the ddPCR successfully detected the L2_UM plasmid successfully.

Using human islet tissue and PBMCs, we were able to detect unmethylated insulin DNA in as low as 0.1 ng of DNA. Also, the ratio of methylated insulin DNA to unmethylated insulin DNA did not change over varying concentrations. We then tested the assay with clinical samples, and were able to detect unmethylated DNA in pre-diabetics. Also, the ratio of unmethylated insulin coding DNA to methylated insulin DNA was significantly elevated in pre-diabetic (mean 0.4264±0.04034 N=6) patients compared to non-diabetic human controls (0.2122±0.02449 N=13).

### Equivalents

While specific embodiments have been discussed, the above specification is intended to be illustrative and not restrictive. Many variations will become apparent to those skilled in the art upon review of this specification.

1. A composition consisting essentially of SEQ ID NO: 1 or SEQ ID NO: 2.

2. A composition of claim 1, which also includes at least one quencher probe.

3. The composition of claim 2, wherein the quencher probe is selected from the group consisting of: fluorescein amidite, Iowa Black FQ quencher and hexachloro-fluorescein.

4. A primer pair consisting essentially of SEQ ID NO: 3 and SEQ ID NO: 4

5. A method of diagnosing whether a subject has or is at risk of developing type 1 diabetes comprising the steps of:
(a) isolating genomic insulin DNA from an appropriate sample obtained from a subject and treating the isolated DNA with bisulfite;
(b) contacting the DNA with an appropriate amount of SEQ ID NOs: 1-4 to form a reaction mixture;
(c) loading the reaction mixture into a droplet generator;
(d) depositing the droplets generated onto a plate and transferring the plate into a polymerase chain reactor for amplification;
(e) transferring the plate into a droplet reader for analysis of the data, wherein the ratio of detected unmethylated insulin DNA copy number to methylated insulin DNA copy number and detection of unmethylated insulin DNA indicates that the subject has or is at risk of developing type 1 diabetes.


SEQUENCE LISTING (419 sequences)

SEQ ID NO: 1 - DNA, 25 bp, Artificial Sequence
  Synthetic primer
atttaagatttgttgggaggtagag

SEQ ID NO: 2 - DNA, 25 bp, Artificial Sequence
  Synthetic primer
atttaagattcgtcgggaggtagag

SEQ ID NO: 3 - DNA, 19 bp, Artificial Sequence
  Synthetic primer
gtggtttatatttggtgga

SEQ ID NO: 4 - DNA, 20 bp, Artificial Sequence
  Synthetic primer
attaactcaccctacaaatc

SEQ ID NO: 5 - DNA, 19 bp, Artificial Sequence
  Synthetic primer
gtggtttatatttggtgga

SEQ ID NO: 6 - DNA, 20 bp, Artificial Sequence
  Synthetic primer
attaactcaccctacaaatc

SEQ ID NO: 7 - DNA, 16 bp, Artificial Sequence
  Synthetic primer
agagttttgttttgta

SEQ ID NO: 8 - DNA, 16 bp, Artificial Sequence
  Synthetic primer
agagtttcgttttgta

SEQ ID NO: 9 - DNA, 15 bp, Artificial Sequence
  Synthetic primer
gggatagtagtgtaa

SEQ ID NO: 10 - DNA, 15 bp, Artificial Sequence
  Synthetic primer
cctactcacaactaa

SEQ ID NO: 11 - DNA, 15 bp, Artificial Sequence
  Synthetic primer
tggttattgggtttt

SEQ ID NO: 12 - DNA, 15 bp, Artificial Sequence
  Synthetic primer
tggttatcgggtttt

SEQ ID NO: 13 - DNA, 24 bp, Artificial Sequence
  Synthetic primer
ggaaagtggtttaggtgagggttt

SEQ ID NO: 14 - DNA, 27 bp, Artificial Sequence
  Synthetic primer
ctccttaatcatcaacacctcttcctc

SEQ ID NO: 15 - DNA, 22 bp, Artificial Sequence
  Synthetic primer
tagttgtgagtagggataggtt

SEQ ID NO: 16 - DNA, 23 bp, Artificial Sequence
  Synthetic primer
aatctccttaatcatcaacacct

SEQ ID NO: 17 - DNA, 24 bp, Artificial Sequence
  Synthetic primer
ggtttaatgtggaaagtggtttag

SEQ ID NO: 18 - DNA, 28 bp, Artificial Sequence
  Synthetic primer
aaccatttccctaatactaaatctataa

SEQ ID NO: 19 - DNA, 20 bp, Artificial Sequence
  Synthetic primer
tttggtttaatgtggaaagt

SEQ ID NO: 20 - DNA, 20 bp, Artificial Sequence
  Synthetic primer
ccaaaccatttccctaatac

SEQ ID NO: 21 - DNA, 21 bp, Artificial Sequence
  Synthetic primer
gggatagtagtgtaaagagtt

SEQ ID NO: 22 - DNA, 22 bp, Artificial Sequence
  Synthetic primer
actacaatttccaaaccatttc

SEQ ID NO: 23 - DNA, 21 bp, Artificial Sequence
  Synthetic primer
ttaatgatttgttggttttga

SEQ ID NO: 24 - DNA, 21 bp, Artificial Sequence
  Synthetic primer
ttaatgattcgttggttttga

SEQ ID NO: 25 - DNA, 24 bp, Artificial Sequence
  Synthetic primer
ggaaagtggtttaggtgagggttt

SEQ ID NO: 26 - DNA, 27 bp, Artificial Sequence
  Synthetic primer
atctccttaatcatcaacacctcttcc

SEQ ID NO: 27 - DNA, 24 bp, Artificial Sequence
  Synthetic primer
gtgagtagggataggtttggttat

SEQ ID NO: 28 - DNA, 22 bp, Artificial Sequence
  Synthetic primer
ccctaccacctaacccattaaa

SEQ ID NO: 29 - DNA, 23 bp, Artificial Sequence
  Synthetic primer
ggataggtttggttattgggttt

SEQ ID NO: 30 - DNA, 26 bp, Artificial Sequence
  Synthetic primer
tccaaaccatttccctaatactaaat

SEQ ID NO: 31 - DNA, 21 bp, Artificial Sequence
  Synthetic primer
tagttgtgagtagggataggt

SEQ ID NO: 32 - DNA, 24 bp, Artificial Sequence
  Synthetic primer
aaactacaatttccaaaccatttc

SEQ ID NO: 33 - DNA, 22 bp, Artificial Sequence
  Synthetic primer
tttaatgtggaaagtggtttag

SEQ ID NO: 34 - DNA, 22 bp, Artificial Sequence
  Synthetic primer
aaatctccttaatcatcaacac

SEQ ID NO: 35 - DNA, 25 bp, Artificial Sequence
  Synthetic primer
aagaggtgttgatgattaaggagat

SEQ ID NO: 36 - DNA, 25 bp, Artificial Sequence
  Synthetic primer
aagaggtgttgacgattaaggagat

SEQ ID NO: 37 - DNA, 24 bp, Artificial Sequence
  Synthetic primer
gtgagtagggataggtttggttat

SEQ ID NO: 38 - DNA, 22 bp, Artificial Sequence
  Synthetic primer
ccctaccacctaacccattaaa

SEQ ID NO: 39 - DNA, 22 bp, Artificial Sequence
  Synthetic primer
gaaagtggtttaggtgagggtt

SEQ ID NO: 40 - DNA, 28 bp, Artificial Sequence
  Synthetic primer
tccaaaccatttccctaatactaaatct

SEQ ID NO: 41 - DNA, 21 bp, Artificial Sequence
  Synthetic primer
tagttgtgagtagggataggt

SEQ ID NO: 42 - DNA, 24 bp, Artificial Sequence
  Synthetic primer
aaactacaatttccaaaccatttc

SEQ ID NO: 43 - DNA, 22 bp, Artificial Sequence
  Synthetic primer
gataggtttggttattgggttt

SEQ ID NO: 44 - DNA, 23 bp, Artificial Sequence
  Synthetic primer
atttccaaaccatttccctaata

SEQ ID NO: 45 - DNA, 19 bp, Artificial Sequence
  Synthetic primer
tttaatgatttgttggttt

SEQ ID NO: 46 - DNA, 20 bp, Artificial Sequence
  Synthetic primer
cccactaactttataatctc

SEQ ID NO: 47 - DNA, 19 bp, Artificial Sequence
  Synthetic primer
aaatggtttggaaattgta

SEQ ID NO: 48 - DNA, 19 bp, Artificial Sequence
  Synthetic primer
aaatggttcggaaattgta

SEQ ID NO: 49 - DNA, 22 bp, Artificial Sequence
  Synthetic primer
tgaggaagaggtgttgatgatt

SEQ ID NO: 50 - DNA, 22 bp, Artificial Sequence
  Synthetic primer
ccctaccacctaacccattaaa

SEQ ID NO: 51 - DNA, 25 bp, Artificial Sequence
  Synthetic primer
agaggtgttgatgattaaggagatt

SEQ ID NO: 52 - DNA, 27 bp, Artificial Sequence
  Synthetic primer
acctcttctaatacaacctatcctaaa

SEQ ID NO: 53 - DNA, 19 bp, Artificial Sequence
  Synthetic primer
agttgtgagtagggatagg

SEQ ID NO: 54 - DNA, 23 bp, Artificial Sequence
  Synthetic primer
cccactaactttataatctcaaa

SEQ ID NO: 55 - DNA, 17 bp, Artificial Sequence
  Synthetic primer
tttgaggaagaggtgtt

SEQ ID NO: 56 - DNA, 20 bp, Artificial Sequence
  Synthetic primer
acctcttctaatacaaccta

SEQ ID NO: 57 - DNA, 21 bp, Artificial Sequence
  Synthetic primer
tttatagatttagtattaggg

SEQ ID NO: 58 - DNA, 19 bp, Artificial Sequence
  Synthetic primer
ctacttaataacctcttct

SEQ ID NO: 59 - DNA, 13 bp, Artificial Sequence
  Synthetic primer
ttaggtggtaggg

SEQ ID NO: 60 - DNA, 13 bp, Artificial Sequence
  Synthetic primer
ttaggtggtaggg

SEQ ID NO: 61 - DNA, 28 bp, Artificial Sequence
  Synthetic primer
aataacctcttctaatacaacctatcct

SEQ ID NO: 62 - DNA, 22 bp, Artificial Sequence
  Synthetic primer
agtattagggaaatggtttgga

SEQ ID NO: 63 - DNA, 27 bp, Artificial Sequence
  Synthetic primer
caaacctacttaataacctcttctaat

SEQ ID NO: 64 - DNA, 18 bp, Artificial Sequence
  Synthetic primer
gaggaagaggtgttgatg

SEQ ID NO: 65 - DNA, 23 bp, Artificial Sequence
  Synthetic primer
cccactaactttataatctcaaa

SEQ ID NO: 66 - DNA, 18 bp, Artificial Sequence
  Synthetic primer
tttagtattagggaaatg

SEQ ID NO: 67 - DNA, 17 bp, Artificial Sequence
  Synthetic primer
acctacttaataacctc

SEQ ID NO: 68 - DNA, 16 bp, Artificial Sequence
  Synthetic primer
aagttagtgggggttt

SEQ ID NO: 69 - DNA, 16 bp, Artificial Sequence
  Synthetic primer
aagttagcgggggttt

SEQ ID NO: 70 - DNA, 22 bp, Artificial Sequence
  Synthetic primer
tttaatgggttaggtggtaggg

SEQ ID NO: 71 - DNA, 27 bp, Artificial Sequence
  Synthetic primer
acctcttctaatacaacctatcctaaa

SEQ ID NO: 72 - DNA, 25 bp, Artificial Sequence
  Synthetic primer
gggaaatggtttggaaattgtagtt

SEQ ID NO: 73 - DNA, 30 bp, Artificial Sequence
  Synthetic primer
acctacttaataacctcttctaatacaacc

SEQ ID NO: 74 - DNA, 24 bp, Artificial Sequence
  Synthetic primer
gtattagggaaatggtttggaaat

SEQ ID NO: 75 - DNA, 26 bp, Artificial Sequence
  Synthetic primer
acaaacctacttaataacctcttcta

SEQ ID NO: 76 - DNA, 17 bp, Artificial Sequence
  Synthetic primer
agtattagggaaatggt

SEQ ID NO: 77 - DNA, 18 bp, Artificial Sequence
  Synthetic primer
caaacctacttaataacc

SEQ ID NO: 78 - DNA, 12 bp, Artificial Sequence
  Synthetic primer
tttgtgttaggt

SEQ ID NO: 79 - DNA, 12 bp, Artificial Sequence
  Synthetic primer
tttgcgttaggt

SEQ ID NO: 80 - DNA, 30 bp, Artificial Sequence
  Synthetic primer
ggttgtattagaagaggttattaagtaggt

SEQ ID NO: 81 - DNA, 22 bp, Artificial Sequence
  Synthetic primer
cttcacaaacccaaccacatcc

SEQ ID NO: 82 - DNA, 30 bp, Artificial Sequence
  Synthetic primer
ttaggataggttgtattagaagaggttatt

SEQ ID NO: 83 - DNA, 20 bp, Artificial Sequence
  Synthetic primer
ccaaccacatcctccctact

SEQ ID NO: 84 - DNA, 20 bp, Artificial Sequence
  Synthetic primer
taatgggttaggtggtaggg

SEQ ID NO: 85 - DNA, 19 bp, Artificial Sequence
  Synthetic primer
cccacatacttcacaaacc

SEQ ID NO: 86 - DNA, 21 bp, Artificial Sequence
  Synthetic primer
aggataggttgtattagaaga

SEQ ID NO: 87 - DNA, 18 bp, Artificial Sequence
  Synthetic primer
aaatccaaccaccctaaa

SEQ ID NO: 88 - DNA, 21 bp, Artificial Sequence
  Synthetic primer
ttgagattataaagttagtgg

SEQ ID NO: 89 - DNA, 17 bp, Artificial Sequence
  Synthetic primer
aaaccaaataccctacc

SEQ ID NO: 90 - DNA, 20 bp, Artificial Sequence
  Synthetic primer
tagggaggatgtggttgggt

SEQ ID NO: 91 - DNA, 20 bp, Artificial Sequence
  Synthetic primer
tagggaggacgtggttgggt

SEQ ID NO: 92 - DNA, 19 bp, Artificial Sequence
  Synthetic primer
gtgttaggtgggtttagga

SEQ ID NO: 93 - DNA, 19 bp, Artificial Sequence
  Synthetic primer
aaaccaacaacaccaacaa

SEQ ID NO: 94 - DNA, 23 bp, Artificial Sequence
  Synthetic primer
aggataggttgtattagaagagg

SEQ ID NO: 95 - DNA, 22 bp, Artificial Sequence
  Synthetic primer
aaaccaaataccctaccttaaa

SEQ ID NO: 96 - DNA, 17 bp, Artificial Sequence
  Synthetic primer
ttagggtggttggattt

SEQ ID NO: 97 - DNA, 22 bp, Artificial Sequence
  Synthetic primer
cacacaaatattaattcacaaa

SEQ ID NO: 98 - DNA, 16 bp, Artificial Sequence
  Synthetic primer
ttgttggtgttgttgg

SEQ ID NO: 99 - DNA, 16 bp, Artificial Sequence
  Synthetic primer
ttgttggcgttgttgg

SEQ ID NO: 100 - DNA, 22 bp, Artificial Sequence
  Synthetic primer
gaggatgtggttgggtttgtga

SEQ ID NO: 101 - DNA, 30 bp, Artificial Sequence
  Synthetic primer
aacttccaccaaatataaaccacacaaata

SEQ ID NO: 102 - DNA, 22 bp, Artificial Sequence
  Synthetic primer
ggttgggtttgtgaagtatgtg

SEQ ID NO: 103 - DNA, 30 bp, Artificial Sequence
  Synthetic primer
accaaatataaaccacacaaatattaattc

SEQ ID NO: 104 - DNA, 20 bp, Artificial Sequence
  Synthetic primer
ttgtagtagggaggatgtgg

SEQ ID NO: 105 - DNA, 23 bp, Artificial Sequence
  Synthetic primer
aaacttccaccaaatataaacca

SEQ ID NO: 106 - DNA, 22 bp, Artificial Sequence
  Synthetic primer
tttaaggtagggtatttggttt

SEQ ID NO: 107 - DNA, 20 bp, Artificial Sequence
  Synthetic primer
cctctacctcccaacaaatc

SEQ ID NO: 108 - DNA, 19 bp, Artificial Sequence
  Synthetic primer
gtgttaggtgggtttagga

SEQ ID NO: 109 - DNA, 23 bp, Artificial Sequence
  Synthetic primer
aaactacaactaaatcaaatccc

SEQ ID NO: 110 - DNA, 22 bp, Artificial Sequence
  Synthetic primer
tttaaggtagggtatttggttt

SEQ ID NO: 111 - DNA, 24 bp, Artificial Sequence
  Synthetic primer
accacacaaatattaattcacaaa

SEQ ID NO: 112 - DNA, 20 bp, Artificial Sequence
  Synthetic primer
ttgtagtagggaggatgtgg

SEQ ID NO: 113 - DNA, 23 bp, Artificial Sequence
  Synthetic primer
aaactacaactaaatcaaatccc

SEQ ID NO: 114 - DNA, 12 bp, Artificial Sequence
  Synthetic primer
ttagttgtagtt

SEQ ID NO: 115 - DNA, 12 bp, Artificial Sequence
  Synthetic primer
ttagtcgtagtt

SEQ ID NO: 116 - DNA, 22 bp, Artificial Sequence
  Synthetic primer
ggttgggtttgtgaagtatgtg

SEQ ID NO: 117 - DNA, 30 bp, Artificial Sequence; identical to SEQ ID NO: 103
  Synthetic primer

SEQ ID NO: 118 - DNA, 20 bp, Artificial Sequence
  Synthetic primer
ttgtagtagggaggatgtgg

SEQ ID NO: 119 - DNA, 23 bp, Artificial Sequence
  Synthetic primer
aaacttccaccaaatataaacca

SEQ ID NO: 120 - DNA, 20 bp, Artificial Sequence
  Synthetic primer
tttgttggtgttgttggttt

SEQ ID NO: 121 - DNA, 20 bp, Artificial Sequence
  Synthetic primer
cctctacctcccaacaaatc

SEQ ID NO: 122 - DNA, 22 bp, Artificial Sequence
  Synthetic primer
tttaaggtagggtatttggttt

SEQ ID NO: 123 - DNA, 24 bp, Artificial Sequence
  Synthetic primer
accacacaaatattaattcacaaa

SEQ ID NO: 124 - DNA, 12 bp, Artificial Sequence
  Synthetic primer
ttgtgtggttta

SEQ ID NO: 125 - DNA, 12 bp, Artificial Sequence
  Synthetic primer
ttgtgcggttta

SEQ ID NO: 126 - DNA, 20 bp, Artificial Sequence
  Synthetic primer
tttgttggtgttgttggttt

SEQ ID NO: 127 - DNA, 20 bp, Artificial Sequence
  Synthetic primer
cctctacctcccaacaaatc

SEQ ID NO: 128 - DNA, 23 bp, Artificial Sequence
  Synthetic primer
gggatttgatttagttgtagttt

SEQ ID NO: 129 - DNA, 21 bp, Artificial Sequence
  Synthetic primer
ctcaccctacaaatcctctac

SEQ ID NO: 130 - DNA, 21 bp, Artificial Sequence
  Synthetic primer
tttaaggtagggtatttggtt

SEQ ID NO: 131 - DNA, 22 bp, Artificial Sequence
  Synthetic primer
acctcccaacaaatcttaaata

SEQ ID NO: 132 - DNA, 17 bp, Artificial Sequence
  Synthetic primer
gttgggtttgtgaagta

SEQ ID NO: 133 - DNA, 17 bp, Artificial Sequence
  Synthetic primer
cccacacactaaataaa

SEQ ID NO: 134 - DNA, 13 bp, Artificial Sequence
  Synthetic primer
agtgtgtggggaa

SEQ ID NO: 135 - DNA, 13 bp, Artificial Sequence
  Synthetic primer
agtgtgcggggaa

SEQ ID NO: 136 - DNA, 28 bp, Artificial Sequence
  Synthetic primer
atttgtgtggtttatatttggtggaagt

SEQ ID NO: 137 - DNA, 24 bp, Artificial Sequence
  Synthetic primer
actcaccctacaaatcctctacct

SEQ ID NO: 138 - DNA, 30 bp, Artificial Sequence
  Synthetic primer
ttgtgaattaatatttgtgtggtttatatt

SEQ ID NO: 139 - DNA, 21 bp, Artificial Sequence
  Synthetic primer
tcctctacctcccaacaaatc

SEQ ID NO: 140 - DNA, 20 bp, Artificial Sequence
  Synthetic primer
tttgttggtgttgttggttt

SEQ ID NO: 141 - DNA, 25 bp, Artificial Sequence
  Synthetic primer
tctacctcccaacaaatcttaaata

SEQ ID NO: 142 - DNA, 24 bp, Artificial Sequence
  Synthetic primer
tttgtgaattaatatttgtgtggt

SEQ ID NO: 143 - DNA, 24 bp, Artificial Sequence
  Synthetic primer
caataaacaattaactcaccctac

SEQ ID NO: 144 - DNA, 23 bp, Artificial Sequence
  Synthetic primer
gggatttgatttagttgtagttt

SEQ ID NO: 145 - DNA, 21 bp, Artificial Sequence
  Synthetic primer
ctaaataacaacctcctaccc

SEQ ID NO: 146 - DNA, 14 bp, Artificial Sequence
  Synthetic primer
agatttgttgggag

SEQ ID NO: 147 - DNA, 14 bp, Artificial Sequence
  Synthetic primer
agattcgtcgggag

SEQ ID NO: 148 - DNA, 28 bp, Artificial Sequence
  Synthetic primer
atttgtgtggtttatatttggtggaagt

SEQ ID NO: 149 - DNA, 29 bp, Artificial Sequence
  Synthetic primer
acaattaactcaccctacaaatcctctac

SEQ ID NO: 150 - DNA, 30 bp, Artificial Sequence
  Synthetic primer
tgaattaatatttgtgtggtttatatttgg

SEQ ID NO: 151 - DNA, 26 bp, Artificial Sequence
  Synthetic primer
acaataaacaattaactcaccctaca

SEQ ID NO: 152 - DNA, 27 bp, Artificial Sequence
  Synthetic primer
tttgtgaattaatatttgtgtggttta

SEQ ID NO: 153 - DNA, 24 bp, Artificial Sequence
  Synthetic primer
cctactaaataacaacctcctacc

SEQ ID NO: 154 - DNA, 18 bp, Artificial Sequence
  Synthetic primer
tgttggtgttgttggttt

SEQ ID NO: 155 - DNA, 23 bp, Artificial Sequence
  Synthetic primer
cccttctacccatactaaataaa

SEQ ID NO: 156 - DNA, 20 bp, Artificial Sequence
  Synthetic primer
tttgtgaattaatatttgtg

SEQ ID NO: 157 - DNA, 18 bp, Artificial Sequence
  Synthetic primer
ccctactaaataacaacc

SEQ ID NO: 158 - DNA, 12 bp, Artificial Sequence
  Synthetic primer
ttggttgttttt

SEQ ID NO: 159 - DNA, 12 bp, Artificial Sequence
  Synthetic primer
ttggtcgttttt

SEQ ID NO: 160 - DNA, 27 bp, Artificial Sequence
  Synthetic primer
ggtagaggatttgtagggtgagttaat

SEQ ID NO: 161 - DNA, 26 bp, Artificial Sequence
  Synthetic primer
ccctactaaataacaacctcctaccc

SEQ ID NO: 162 - DNA, 24 bp, Artificial Sequence
  Synthetic primer
ttgttgggaggtagaggatttgta

SEQ ID NO: 163 - DNA, 25 bp, Artificial Sequence
  Synthetic primer
aaaccaacaccatcctcaaactaaa

SEQ ID NO: 164 - DNA, 28 bp, Artificial Sequence
  Synthetic primer
agggtgagttaattgtttattgttgttt

SEQ ID NO: 165 - DNA, 27 bp, Artificial Sequence
  Synthetic primer
acatacccaccctctaatatatctcaa

SEQ ID NO: 166 - DNA, 22 bp, Artificial Sequence
  Synthetic primer
taagatttgttgggaggtagag

SEQ ID NO: 167 - DNA, 23 bp, Artificial Sequence
  Synthetic primer
cccttctacccatactaaataaa

SEQ ID NO: 168 - DNA, 23 bp, Artificial Sequence
  Synthetic primer
gtagggtgagttaattgtttatt

SEQ ID NO: 169 - DNA, 17 bp, Artificial Sequence
  Synthetic primer
aaccaacaccatcctca

SEQ ID NO: 170 - DNA, 12 bp, Artificial Sequence
  Synthetic primer
tttggtgttttt

SEQ ID NO: 171 - DNA, 12 bp, Artificial Sequence
  Synthetic primer
tttggcgttttt

SEQ ID NO: 172 - DNA, 27 bp, Artificial Sequence
  Synthetic primer
ggtagaggatttgtagggtgagttaat

SEQ ID NO: 173 - DNA, 26 bp, Artificial Sequence
  Synthetic primer
ccctactaaataacaacctcctaccc

SEQ ID NO: 174 - DNA, 24 bp, Artificial Sequence
  Synthetic primer
ttgttgggaggtagaggatttgta

SEQ ID NO: 175 - DNA, 25 bp, Artificial Sequence
  Synthetic primer
aaaccaacaccatcctcaaactaaa

SEQ ID NO: 176 - DNA, 28 bp, Artificial Sequence
  Synthetic primer
agggtgagttaattgtttattgttgttt

SEQ ID NO: 177 - DNA, 27 bp, Artificial Sequence
  Synthetic primer
acatacccaccctctaatatatctcaa

SEQ ID NO: 178 - DNA, 25 bp, Artificial Sequence
  Synthetic primer
atttaagatttgttgggaggtagag

SEQ ID NO: 179 - DNA, 25 bp, Artificial Sequence
  Synthetic primer
atttaagattcgtcgggaggtagag

SEQ ID NO: 180 - DNA, 20 bp, Artificial Sequence
  Synthetic primer
agatttgttgggaggtagag

SEQ ID NO: 181 - DNA, 18 bp, Artificial Sequence
  Synthetic primer
aaaccaacaccatcctca

SEQ ID NO: 182 - DNA, 22 bp, Artificial Sequence
  Synthetic primer
gtagggtgagttaattgtttat

SEQ ID NO: 183 - DNA, 20 bp, Artificial Sequence
  Synthetic primer
cccttctacccatactaaat

SEQ ID NO: 184 - DNA, 14 bp, Artificial Sequence
  Synthetic primer
tggttatgttttaa

SEQ ID NO: 185 - DNA, 14 bp, Artificial Sequence
  Synthetic primer
tggttacgttttaa

SEQ ID NO: 186 - DNA, 26 bp, Artificial Sequence
  Synthetic primer
gggtaggaggttgttatttagtaggg

SEQ ID NO: 187 - DNA, 22 bp, Artificial Sequence
  Synthetic primer
aaaccaacaccatcctcaaact

SEQ ID NO: 188 - DNA, 28 bp, Artificial Sequence
  Synthetic primer
agggtgagttaattgtttattgttgttt

SEQ ID NO: 189 - DNA, 27 bp, Artificial Sequence
  Synthetic primer
acatacccaccctctaatatatctcaa

SEQ ID NO: 190 - DNA, 19 bp, Artificial Sequence
  Synthetic primer
gggtaggaggttgttattt

SEQ ID NO: 191 - DNA, 19 bp, Artificial Sequence
  Synthetic primer
acatacccaccctctaata

SEQ ID NO: 192 - DNA, 18 bp, Artificial Sequence
  Synthetic primer
ttagtatgggtagaaggg

SEQ ID NO: 193 - DNA, 21 bp, Artificial Sequence
  Synthetic primer
aaattctaactaaaccacaaa

SEQ ID NO: 194 - DNA, 14 bp, Artificial Sequence
  Synthetic primer
tgaggatggtgttg

SEQ ID NO: 195 - DNA, 14 bp, Artificial Sequence
  Synthetic primer
tgaggacggtgttg

SEQ ID NO: 196 - DNA, 28 bp, Artificial Sequence
  Synthetic primer
agggtgagttaattgtttattgttgttt

SEQ ID NO: 197 - DNA, 27 bp, Artificial Sequence
  Synthetic primer
acatacccaccctctaatatatctcaa

SEQ ID NO: 198 - DNA, 22 bp, Artificial Sequence
  Synthetic primer
gggtaggaggttgttatttagt

SEQ ID NO: 199 - DNA, 21 bp, Artificial Sequence
  Synthetic primer
aaacatacccaccctctaata

SEQ ID NO: 200 - DNA, 21 bp, Artificial Sequence
  Synthetic primer
tttgtggtttagttagaattt

SEQ ID NO: 201 - DNA, 17 bp, Artificial Sequence
  Synthetic primer
ccctcctccaaacataa

SEQ ID NO: 202 - DNA, 28 bp, Artificial Sequence
  Synthetic primer
agggtgagttaattgtttattgttgttt

SEQ ID NO: 203 - DNA, 27 bp, Artificial Sequence
  Synthetic primer
acatacccaccctctaatatatctcaa

SEQ ID NO: 204 - DNA, 22 bp, Artificial Sequence
  Synthetic primer
gggtaggaggttgttatttagt

SEQ ID NO: 205 - DNA, 21 bp, Artificial Sequence
  Synthetic primer
aaacatacccaccctctaata

SEQ ID NO: 206 - DNA, 18 bp, Artificial Sequence
  Synthetic primer
ttagtttgaggatggtgt

SEQ ID NO: 207 - DNA, 20 bp, Artificial Sequence
  Synthetic primer
cctcctccaaacataataaa

SEQ ID NO: 208 - DNA, 21 bp, Artificial Sequence
  Synthetic primer
tttgtggtttagttagaattt

SEQ ID NO: 209 - DNA, 22 bp, Artificial Sequence
  Synthetic primer
aaacacttaaatctacaatcat

SEQ ID NO: 210 - DNA, 12 bp, Artificial Sequence
  Synthetic primer
agttttgagata

SEQ ID NO: 211 - DNA, 12 bp, Artificial Sequence
  Synthetic primer
agtttcgagata

SEQ ID NO: 212 - DNA, 19 bp, Artificial Sequence
  Synthetic primer
ttgaggatggtgttggttt

SEQ ID NO: 213 - DNA, 22 bp, Artificial Sequence
  Synthetic primer
caccctcctccaaacataataa

SEQ ID NO: 214 - DNA, 22 bp, Artificial Sequence
  Synthetic primer
gggtaggaggttgttatttagt

SEQ ID NO: 215 - DNA, 21 bp, Artificial Sequence
  Synthetic primer
aaacatacccaccctctaata

SEQ ID NO: 216 - DNA, 18 bp, Artificial Sequence
  Synthetic primer
tttagtttgaggatggtg

SEQ ID NO: 217 - DNA, 22 bp, Artificial Sequence
  Synthetic primer
aaacacttaaatctacaatcat

SEQ ID NO: 218 - DNA, 19 bp, Artificial Sequence
  Synthetic primer
tgtggtttagttagaattt

SEQ ID NO: 219 - DNA, 20 bp, Artificial Sequence
  Synthetic primer
aatctacaatcatcaaataa

SEQ ID NO: 220 - DNA, 13 bp, Artificial Sequence
  Synthetic primer
tgggtatgttttt

SEQ ID NO: 221 - DNA, 13 bp, Artificial Sequence
  Synthetic primer
tgggtacgttttt

SEQ ID NO: 222 - DNA, 19 bp, Artificial Sequence
  Synthetic primer
ttgaggatggtgttggttt

SEQ ID NO: 223 - DNA, 22 bp, Artificial Sequence
  Synthetic primer
caccctcctccaaacataataa

SEQ ID NO: 224 - DNA, 25 bp, Artificial Sequence
  Synthetic primer
gggtaggaggttgttatttagtagg

SEQ ID NO: 225 - DNA, 30 bp, Artificial Sequence
  Synthetic primer
aaacacttaaatctacaatcatcaaataaa

SEQ ID NO: 226 - DNA, 18 bp, Artificial Sequence
  Synthetic primer
gggtaggaggttgttatt

SEQ ID NO: 227 - DNA, 22 bp, Artificial Sequence
  Synthetic primer
aaacacttaaatctacaatcat

SEQ ID NO: 228 - DNA, 18 bp, Artificial Sequence
  Synthetic primer
tttagtttgaggatggtg

SEQ ID NO: 229 - DNA, 17 bp, Artificial Sequence
  Synthetic primer
caaacaaacaaccacac

SEQ ID NO: 230 - DNA, 12 bp, Artificial Sequence
  Synthetic primer
ttatttgttttt

SEQ ID NO: 231 - DNA, 12 bp, Artificial Sequence
  Synthetic primer
ttattcgttttt

SEQ ID NO: 232 - DNA, 28 bp, Artificial Sequence
  Synthetic primer
tttgagatatattagagggtgggtatgt

SEQ ID NO: 233 - DNA, 22 bp, Artificial Sequence
  Synthetic primer
aacccactcaaacaaacaacca

SEQ ID NO: 234 - DNA, 19 bp, Artificial Sequence
  Synthetic primer
ttgaggatggtgttggttt

SEQ ID NO: 235 - DNA, 22 bp, Artificial Sequence
  Synthetic primer
caccctcctccaaacataataa

SEQ ID NO: 236 - DNA, 25 bp, Artificial Sequence
  Synthetic primer
gggtaggaggttgttatttagtagg

SEQ ID NO: 237 - DNA, 30 bp, Artificial Sequence; identical to SEQ ID NO: 225
  Synthetic primer

SEQ ID NO: 238 - DNA, 18 bp, Artificial Sequence
  Synthetic primer
gggtaggaggttgttatt

SEQ ID NO: 239 - DNA, 22 bp, Artificial Sequence
  Synthetic primer
aaacacttaaatctacaatcat

SEQ ID NO: 240 - DNA, 12 bp, Artificial Sequence
  Synthetic primer
tgttttgtagtt

SEQ ID NO: 241 - DNA, 12 bp, Artificial Sequence
  Synthetic primer
tgtttcgtagtt

SEQ ID NO: 242 - DNA, 26 bp, Artificial Sequence
  Synthetic primer
gagatatattagagggtgggtatgtt

SEQ ID NO: 243 - DNA, 24 bp, Artificial Sequence
  Synthetic primer
acaccctcctccaaacataataaa

SEQ ID NO: 244 - DNA, 22 bp, Artificial Sequence
  Synthetic primer
gggatagtagtgtaaagagttt

SEQ ID NO: 245 - DNA, 22 bp, Artificial Sequence
  Synthetic primer
cctatccctactcacaactaaa

SEQ ID NO: 246 - DNA, 17 bp, Artificial Sequence
  Synthetic primer
tgaggatggtgttggtt

SEQ ID NO: 247 - DNA, 27 bp, Artificial Sequence
  Synthetic primer
cacttaaatctacaatcatcaaataaa

SEQ ID NO: 248 - DNA, 21 bp, Artificial Sequence
  Synthetic primer
tttgtggtttagttagaattt

SEQ ID NO: 249 - DNA, 22 bp, Artificial Sequence
  Synthetic primer
aaacacttaaatctacaatcat

SEQ ID NO: 250 - DNA, 11 bp, Artificial Sequence
  Synthetic primer
atgattgtaga

SEQ ID NO: 251 - DNA, 11 bp, Artificial Sequence
  Synthetic primer
atgatcgtaga

SEQ ID NO: 252 - DNA, 26 bp, Artificial Sequence
  Synthetic primer
gagatatattagagggtgggtatgtt

SEQ ID NO: 253 - DNA, 24 bp, Artificial Sequence
  Synthetic primer
acaccctcctccaaacataataaa

SEQ ID NO: 254 - DNA, 17 bp, Artificial Sequence
  Synthetic primer
tgttttatgttgtttgt

SEQ ID NO: 255 - DNA, 17 bp, Artificial Sequence
  Synthetic primer
tgttttacgttgtttgt

SEQ ID NO: 256 - DNA, 26 bp, Artificial Sequence
  Synthetic primer
gagatatattagagggtgggtatgtt

SEQ ID NO: 257 - DNA, 24 bp, Artificial Sequence
  Synthetic primer
acaccctcctccaaacataataaa

SEQ ID NO: 258 - DNA, 30 bp, Artificial Sequence
  Synthetic primer
tttatttgatgattgtagatttaagtgttt

SEQ ID NO: 259 - DNA, 18 bp, Artificial Sequence
  Synthetic primer
aaacaaccacaccctcct

SEQ ID NO: 260 - DNA, 20 bp, Artificial Sequence
  Synthetic primer
tatttgatgattgtagattt

SEQ ID NO: 261 - DNA, 18 bp, Artificial Sequence
  Synthetic primer
cccatctcctaactataa

SEQ ID NO: 262 - DNA, 12 bp, Artificial Sequence
  Synthetic primer
ttgggtgaatat

SEQ ID NO: 263 - DNA, 12 bp, Artificial Sequence
  Synthetic primer
ttgggcgaatat

SEQ ID NO: 264 - DNA, 26 bp, Artificial Sequence
  Synthetic primer
gagatatattagagggtgggtatgtt

SEQ ID NO: 265 - DNA, 24 bp, Artificial Sequence
  Synthetic primer
acaccctcctccaaacataataaa

SEQ ID NO: 266 - DNA, 30 bp, Artificial Sequence; identical to SEQ ID NO: 258
  Synthetic primer

SEQ ID NO: 267 - DNA, 18 bp, Artificial Sequence
  Synthetic primer
aaacaaccacaccctcct

SEQ ID NO: 268 - DNA, 20 bp, Artificial Sequence
  Synthetic primer
tatttgatgattgtagattt

SEQ ID NO: 269 - DNA, 18 bp, Artificial Sequence
  Synthetic primer
cccatctcctaactataa

SEQ ID NO: 270 - DNA, 14 bp, Artificial Sequence
  Synthetic primer
attatgtttggagg

SEQ ID NO: 271 - DNA, 14 bp, Artificial Sequence
  Synthetic primer
attacgttcggagg

SEQ ID NO: 272 - DNA, 30 bp, Artificial Sequence; identical to SEQ ID NO: 258
  Synthetic primer

SEQ ID NO: 273 - DNA, 20 bp, Artificial Sequence
  Synthetic primer
caaacaaacaaccacaccct

SEQ ID NO: 274 - DNA, 20 bp, Artificial Sequence
  Synthetic primer
tatttgatgattgtagattt

SEQ ID NO: 275 - DNA, 18 bp, Artificial Sequence
  Synthetic primer
cccatctcctaactataa

SEQ ID NO: 276 - DNA, 15 bp, Artificial Sequence
  Synthetic primer
aggagggtgtggttg

SEQ ID NO: 277 - DNA, 15 bp, Artificial Sequence
  Synthetic primer
aggagggcgtggttg

SEQ ID NO: 278 - DNA, 25 bp, Artificial Sequence
  Synthetic primer
ttgatgattgtagatttaagtgttt

SEQ ID NO: 279 - DNA, 22 bp, Artificial Sequence
  Synthetic primer
aaatctaacccactcaaacaaa

SEQ ID NO: 280 - DNA, 20 bp, Artificial Sequence
  Synthetic primer
tatttgatgattgtagattt

SEQ ID NO: 281 - DNA, 18 bp, Artificial Sequence
  Synthetic primer
cccatctcctaactataa

SEQ ID NO: 282 - DNA, 13 bp, Artificial Sequence
  Synthetic primer
ttttgttgttagg

SEQ ID NO: 283 - DNA, 13 bp, Artificial Sequence
  Synthetic primer
ttttgtcgttagg

SEQ ID NO: 284 - DNA, 25 bp, Artificial Sequence
  Synthetic primer
tggttgtttgtttgagtgggttaga

SEQ ID NO: 285 - DNA, 21 bp, Artificial Sequence
  Synthetic primer
acccacatatcccacctcctt

SEQ ID NO: 286 - DNA, 20 bp, Artificial Sequence
  Synthetic primer
tttggaggagggtgtggttg

SEQ ID NO: 287 - DNA, 30 bp, Artificial Sequence
  Synthetic primer
aacctaaacaaataatcccaatacttctcc

SEQ ID NO: 288 - DNA, 22 bp, Artificial Sequence
  Synthetic primer
gagggtgtggttgtttgtttga

SEQ ID NO: 289 - DNA, 20 bp, Artificial Sequence
  Synthetic primer
cccaacacccacatatccca

SEQ ID NO: 290 - DNA, 18 bp, Artificial Sequence
  Synthetic primer
ttatgtttggaggagggt

SEQ ID NO: 291 - DNA, 24 bp, Artificial Sequence
  Synthetic primer
aaacctaaacaaataatcccaata

SEQ ID NO: 292 - DNA, 21 bp, Artificial Sequence
  Synthetic primer
tgattgtagatttaagtgttt

SEQ ID NO: 293 - DNA, 19 bp, Artificial Sequence
  Synthetic primer
cccatctcctaactataaa

SEQ ID NO: 294 - DNA, 11 bp, Artificial Sequence
  Synthetic primer
ttttatggtag

SEQ ID NO: 295 - DNA, 11 bp, Artificial Sequence
  Synthetic primer
ttttacggtag

SEQ ID NO: 296 - DNA, 25 bp, Artificial Sequence
  Synthetic primer
tggttgtttgtttgagtgggttaga

SEQ ID NO: 297 - DNA, 21 bp, Artificial Sequence
  Synthetic primer
acccacatatcccacctcctt

SEQ ID NO: 298 - DNA, 20 bp, Artificial Sequence
  Synthetic primer
tttggaggagggtgtggttg

SEQ ID NO: 299 - DNA, 30 bp, Artificial Sequence; identical to SEQ ID NO: 287
  Synthetic primer

SEQ ID NO: 300 - DNA, 22 bp, Artificial Sequence
  Synthetic primer
gagggtgtggttgtttgtttga

SEQ ID NO: 301 - DNA, 20 bp, Artificial Sequence
  Synthetic primer
cccaacacccacatatccca

SEQ ID NO: 302 - DNA, 18 bp, Artificial Sequence
  Synthetic primer
ttatgtttggaggagggt

SEQ ID NO: 303 - DNA, 24 bp, Artificial Sequence
  Synthetic primer
aaacctaaacaaataatcccaata

SEQ ID NO: 304 - DNA, 21 bp, Artificial Sequence
  Synthetic primer
tgattgtagatttaagtgttt

SEQ ID NO: 305 - DNA, 19 bp, Artificial Sequence
  Synthetic primer
cccatctcctaactataaa

SEQ ID NO: 306 - DNA, 13 bp, Artificial Sequence
  Synthetic primer
tgtgggtgttggg

SEQ ID NO: 307 - DNA, 13 bp, Artificial Sequence
  Synthetic primer
tgtgggcgttggg

SEQ ID NO: 308 - DNA, 30 bp, Artificial Sequence
  Synthetic primer
gggagaagtattgggattatttgtttaggt

SEQ ID NO: 309 - DNA, 20 bp, Artificial Sequence
  Synthetic primer
acactcccacccatctccaa

SEQ ID NO: 310 - DNA, 17 bp, Artificial Sequence
  Synthetic primer
gggaaggaggtgggata

SEQ ID NO: 311 - DNA, 21 bp, Artificial Sequence
  Synthetic primer
ccatctccaaccaaactaaac

SEQ ID NO: 312 - DNA, 23 bp, Artificial Sequence
  Synthetic primer
gggagaagtattgggattatttg

SEQ ID NO: 313 - DNA, 20 bp, Artificial Sequence
  Synthetic primer
ctacccaccaaccctaaatc

SEQ ID NO: 314 - DNA, 19 bp, Artificial Sequence
  Synthetic primer
tttgtttgagtgggttaga

SEQ ID NO: 315 - DNA, 23 bp, Artificial Sequence
  Synthetic primer
ccacactaaatataaacctacaa

SEQ ID NO: 316 - DNA, 19 bp, Artificial Sequence
  Synthetic primer
tttatagttaggagatggg

SEQ ID NO: 317 - DNA, 19 bp, Artificial Sequence
  Synthetic primer
ccaaactaaacccaaatta

SEQ ID NO: 318 - DNA, 13 bp, Artificial Sequence
  Synthetic primer
ttagtttggttgg

SEQ ID NO: 319 - DNA, 13 bp, Artificial Sequence
  Synthetic primer
ttagttcggttgg

SEQ ID NO: 320 - DNA, 21 bp, Artificial Sequence
  Synthetic primer
ggaggtgggatatgtgggtgt

SEQ ID NO: 321 - DNA, 23 bp, Artificial Sequence
  Synthetic primer
acctacccaccaaccctaaatca

SEQ ID NO: 322 - DNA, 30 bp, Artificial Sequence; identical to SEQ ID NO: 308
  Synthetic primer

SEQ ID NO: 323 - DNA, 22 bp, Artificial Sequence
  Synthetic primer
acaatacccacctacccaccaa

SEQ ID NO: 324 - DNA, 30 bp, Artificial Sequence
  Synthetic primer
gtttgtaggtttatatttagtgtgggtgat

SEQ ID NO: 325 - DNA, 23 bp, Artificial Sequence
  Synthetic primer
ctaaatcacactcccacccatct

SEQ ID NO: 326 - DNA, 17 bp, Artificial Sequence
  Synthetic primer
gggaaggaggtgggata

SEQ ID NO: 327 - DNA, 19 bp, Artificial Sequence
  Synthetic primer
accctaaatcacactccca

SEQ ID NO: 328 - DNA, 19 bp, Artificial Sequence
  Synthetic primer
gggagaagtattgggatta

SEQ ID NO: 329 - DNA, 18 bp, Artificial Sequence
  Synthetic primer
aaacacaatacccaccta

SEQ ID NO: 330 - DNA, 11 bp, Artificial Sequence
  Synthetic primer
agtgtgattta

SEQ ID NO: 331 - DNA, 11 bp, Artificial Sequence
  Synthetic primer
agtgcgattta

SEQ ID NO: 332 - DNA, 30 bp, Artificial Sequence; identical to SEQ ID NO: 308
  Synthetic primer

SEQ ID NO: 333 - DNA, 22 bp, Artificial Sequence
  Synthetic primer
acaatacccacctacccaccaa

SEQ ID NO: 334 - DNA, 25 bp, Artificial Sequence
  Synthetic primer
aatttgggtttagtttggttggaga

SEQ ID NO: 335 - DNA, 11 bp, Artificial Sequence
  Synthetic primer
agtgtgattta

SEQ ID NO: 336 - DNA, 19 bp, Artificial Sequence
  Synthetic primer
cacccaactccacctaccc

SEQ ID NO: 337 - DNA, 21 bp, Artificial Sequence
  Synthetic primer
tttaatttgggtttagtttgg

SEQ ID NO: 338 - DNA, 11 bp, Artificial Sequence
  Synthetic primer
agtgtgattta

SEQ ID NO: 339 - DNA, 18 bp, Artificial Sequence
  Synthetic primer
aacacaatacccacctac

SEQ ID NO: 340 - DNA, 11 bp, Artificial Sequence
  Synthetic primer
ttggtgggtag

SEQ ID NO: 341 - DNA, 11 bp, Artificial Sequence
  Synthetic primer
ttggcgggtag

SEQ ID NO: 342 - DNA, 23 bp, Artificial Sequence
  Synthetic primer
gagatgggtgggagtgtgattta

SEQ ID NO: 343 - DNA, 19 bp, Artificial Sequence
  Synthetic primer
cacccaactccacctaccc

SEQ ID NO: 344 - DNA, 19 bp, Artificial Sequence
  Synthetic primer
ttggagatgggtgggagtg

SEQ ID NO: 345 - DNA, 24 bp, Artificial Sequence
  Synthetic primer
aaactacaaactacctacaccaaa

SEQ ID NO: 346 - DNA, 9 bp, Artificial Sequence
  Synthetic primer
aggtgggta

SEQ ID NO: 347 - DNA, 9 bp, Artificial Sequence
  Synthetic primer
aggcgggta

SEQ ID NO: 348 - DNA, 23 bp, Artificial Sequence
  Synthetic primer
gagatgggtgggagtgtgattta

SEQ ID NO: 349 - DNA, 19 bp, Artificial Sequence
  Synthetic primer
cacccaactccacctaccc

SEQ ID NO: 350 - DNA, 19 bp, Artificial Sequence
  Synthetic primer
ttggagatgggtgggagtg

SEQ ID NO: 351 - DNA, 24 bp, Artificial Sequence
  Synthetic primer
aaactacaaactacctacaccaaa

SEQ ID NO: 352 - DNA, 18 bp, Artificial Sequence
  Synthetic primer
gatttagggttggtgggt

SEQ ID NO: 353 - DNA, 21 bp, Artificial Sequence
  Synthetic primer
ccacaataccacacttctaca

SEQ ID NO: 354 - DNA, 15 bp, Artificial Sequence
  Synthetic primer
tgttttgttgttgtt

SEQ ID NO: 355 - DNA, 15 bp, Artificial Sequence
  Synthetic primer
tgtttcgtcgttgtt

SEQ ID NO: 356 - DNA, 23 bp, Artificial Sequence
  Synthetic primer
gagatgggtgggagtgtgattta

SEQ ID NO: 357 - DNA, 19 bp, Artificial Sequence
  Synthetic primer
cacccaactccacctaccc

SEQ ID NO: 358 - DNA, 20 bp, Artificial Sequence
  Synthetic primer
tttagggttggtgggtaggt

SEQ ID NO: 359 - DNA, 25 bp, Artificial Sequence
  Synthetic primer
ttccacaataccacacttctacaaa

SEQ ID NO: 360 - DNA, 19 bp, Artificial Sequence
  Synthetic primer
ttggagatgggtgggagtg

SEQ ID NO: 361 - DNA, 24 bp, Artificial Sequence
  Synthetic primer
aaactacaaactacctacaccaaa

SEQ ID NO: 362 - DNA, 19 bp, Artificial Sequence
  Synthetic primer
gtaggtgggtattgtgttt

SEQ ID NO: 363 - DNA, 23 bp, Artificial Sequence
  Synthetic primer
ctaatacaacattattccacaat

SEQ ID NO: 364 - DNA, 11 bp, Artificial Sequence
  Synthetic primer
tgttttggaat

SEQ ID NO: 365 - DNA, 11 bp, Artificial Sequence
  Synthetic primer
tgtttcggaat

SEQ ID NO: 366 - DNA, 23 bp, Artificial Sequence
  Synthetic primer
gagatgggtgggagtgtgattta

SEQ ID NO: 367 - DNA, 19 bp, Artificial Sequence
  Synthetic primer
cacccaactccacctaccc

SEQ ID NO: 368 - DNA, 20 bp, Artificial Sequence
  Synthetic primer
tttagggttggtgggtaggt

SEQ ID NO: 369 - DNA, 25 bp, Artificial Sequence
  Synthetic primer
ttccacaataccacacttctacaaa

SEQ ID NO: 370 - DNA, 19 bp, Artificial Sequence
  Synthetic primer
ttggagatgggtgggagtg

SEQ ID NO: 371 - DNA, 24 bp, Artificial Sequence
  Synthetic primer
aaactacaaactacctacaccaaa

SEQ ID NO: 372 - DNA, 19 bp, Artificial Sequence
  Synthetic primer
gtaggtgggtattgtgttt

SEQ ID NO: 373 - DNA, 23 bp, Artificial Sequence
  Synthetic primer
ctaatacaacattattccacaat

SEQ ID NO: 374 - DNA, 18 bp, Artificial Sequence
  Synthetic primer
ttttgtgtggtatgtttt

SEQ ID NO: 375 - DNA, 18 bp, Artificial Sequence
  Synthetic primer
ttttgcgcggtatgtttt

SEQ ID NO: 376 - DNA, 23 bp, Artificial Sequence
  Synthetic primer
gagatgggtgggagtgtgattta

SEQ ID NO: 377 - DNA, 19 bp, Artificial Sequence
  Synthetic primer
cacccaactccacctaccc

SEQ ID NO: 378 - DNA, 20 bp, Artificial Sequence
  Synthetic primer
tttagggttggtgggtaggt

SEQ ID NO: 379 - DNA, 25 bp, Artificial Sequence
  Synthetic primer
ttccacaataccacacttctacaaa

SEQ ID NO: 380 - DNA, 19 bp, Artificial Sequence
  Synthetic primer
ttggagatgggtgggagtg

SEQ ID NO: 381 - DNA, 24 bp, Artificial Sequence
  Synthetic primer
aaactacaaactacctacaccaaa

SEQ ID NO: 382 - DNA, 19 bp, Artificial Sequence
  Synthetic primer
gtaggtgggtattgtgttt

SEQ ID NO: 383 - DNA, 23 bp, Artificial Sequence
  Synthetic primer
ctaatacaacattattccacaat

SEQ ID NO: 384 - DNA, 12 bp, Artificial Sequence
  Synthetic primer
ttgggtgggggt

SEQ ID NO: 385 - DNA, 12 bp, Artificial Sequence
  Synthetic primer
ttgggcgggggt

SEQ ID NO: 386 - DNA, 20 bp, Artificial Sequence
  Synthetic primer
tttagggttggtgggtaggt

SEQ ID NO: 387 - DNA, 25 bp, Artificial Sequence
  Synthetic primer
ttccacaataccacacttctacaaa

SEQ ID NO: 388 - DNA, 19 bp, Artificial Sequence
  Synthetic primer
gatgggtgggagtgtgatt

SEQ ID NO: 389 - DNA, 24 bp, Artificial Sequence
  Synthetic primer
aaactacaaactacctacaccaaa

SEQ ID NO: 390 - DNA, 19 bp, Artificial Sequence
  Synthetic primer
gtaggtgggtattgtgttt

SEQ ID NO: 391 - DNA, 23 bp, Artificial Sequence
  Synthetic primer
ctaatacaacattattccacaat

SEQ ID NO: 392 - DNA, 13 bp, Artificial Sequence
  Synthetic primer
tagaagtgtggta

SEQ ID NO: 393 - DNA, 13 bp, Artificial Sequence
  Synthetic primer
tagaagcgtggta

SEQ ID NO: 394 - DNA, 24 bp, Artificial Sequence
  Synthetic primer
tttggtgtaggtagtttgtagttt

SEQ ID NO: 395 - DNA, 26 bp, Artificial Sequence
  Synthetic primer
aactttattccatctctctcaataca

SEQ ID NO: 396 - DNA, 18 bp, Artificial Sequence
  Synthetic primer
gggtaggtggagttgggt

SEQ ID NO: 397 - DNA, 30 bp, Artificial Sequence
  Synthetic primer
aacaaatactaatacaacattattccacaa

SEQ ID NO: 398 - DNA, 17 bp, Artificial Sequence
  Synthetic primer
tttggtgtaggtagttt

SEQ ID NO: 399 - DNA, 22 bp, Artificial Sequence
  Synthetic primer
caataattctccaactaataaa

SEQ ID NO: 400 - DNA, 13 bp, Artificial Sequence
  Synthetic primer
attagatgtagtt

SEQ ID NO: 401 - DNA, 13 bp, Artificial Sequence
  Synthetic primer
attagacgtagtt

SEQ ID NO: 402 - DNA, 27 bp, Artificial Sequence
  Synthetic primer
tgtagaagtgtggtattgtggaataat

SEQ ID NO: 403 - DNA, 29 bp, Artificial Sequence
  Synthetic primer
aaactttattccatctctctcaatacaaa

SEQ ID NO: 404 - DNA, 21 bp, Artificial Sequence
  Synthetic primer
tttgtagaagtgtggtattgt

SEQ ID NO: 405 - DNA, 22 bp, Artificial Sequence
  Synthetic primer
aaactttattccatctctctca

SEQ ID NO: 406 - DNA, 11 bp, Artificial Sequence
  Synthetic primer
tagtttgtagg

SEQ ID NO: 407 - DNA, 11 bp, Artificial Sequence
  Synthetic primer
tagttcgtagg

SEQ ID NO: 408 - DNA, 27 bp, Artificial Sequence
  Synthetic primer
tgtagaagtgtggtattgtggaataat

SEQ ID NO: 409 - DNA, 29 bp, Artificial Sequence
  Synthetic primer
aaactttattccatctctctcaatacaaa

SEQ ID NO: 410 - DNA, 21 bp, Artificial Sequence
  Synthetic primer
tttgtagaagtgtggtattgt

SEQ ID NO: 411 - DNA, 22 bp, Artificial Sequence
  Synthetic primer
aaactttattccatctctctca

SEQ ID NO: 412 - DNA, 12 bp, Artificial Sequence
  Synthetic primer
atttgttgtttt

SEQ ID NO: 413 - DNA, 12 bp, Artificial Sequence
  Synthetic primer
attcgtcgtttt

SEQ ID NO: 414 - DNA, 26 bp, Artificial Sequence
  Synthetic primer
gtagaagtgtggtattgtggaataat

SEQ ID NO: 415 - DNA, 27 bp, Artificial Sequence
  Synthetic primer
aaactttattccatctctctcaataca

SEQ ID NO: 416 - DNA, 19 bp, Artificial Sequence
  Synthetic primer
ttgtagaagtgtggtattg

SEQ ID NO: 417 - DNA, 20 bp, Artificial Sequence
  Synthetic primer
aaactttattccatctctct

SEQ ID NO: 418 - DNA, 330 bp, Artificial Sequence
  bisulfite-treated methylated human insulin gene
atgtgggggtgagtttaggggttttaaggtagggtatttggtttttagtttgttttagttttgtttgttttttagattattgtttttttgttatggttttgtggatgtgttttttgtttttgttggtgttgttggttttttggggatttgatttagttgtagtttttgtgaattaatatttgtgtggtttatatttggtggaagttttttatttagtgtgtggggaatgaggttttttttatatatttaagatttgttgggaggtagaggatttgtagggtgagttaattgtttattgttgtttttggttgtttttagttattttttgtt

SEQ ID NO: 419 - DNA, 330 bp, Artificial Sequence
  bisulfite-treated un-methylated human insulin gene
atgtgggggtgagtttaggggttttaaggtagggtatttggtttttagtttgttttagttttgtttgttttttagattattgtttttttgttatggttttgtggatgcgttttttgtttttgttggcgttgttggttttttggggatttgatttagtcgtagtttttgtgaattaatatttgtgcggtttatatttggtggaagttttttatttagtgtgcggggaacgaggttttttttatatatttaagattcgtcgggaggtagaggatttgtagggtgagttaattgtttattgttgtttttggtcgtttttagttattttttgtt